(19) United States
(12) Patent Application Publication
(10) Pub. No.: US 2009/0053306 A1
(43) Pub. Date: Feb. 26, 2009

(54) Pharmaceutical Compositions of a 5-HT2A Serotonin Receptor Modulator Useful for the Treatment of Disorders Related Thereto

(76) Inventors: Rajesh K. Agarwal, San Diego, CA (US); Rampurna Prasad Gullapalli, San Bruno, CA (US); Michael Martin, Carlsbad, CA (US); Michael Morgan, San Diego, CA (US); Dennis Chapman, San Deigo, CA (US); Joseph Belier Speer, Ramona, CA (US); Yun Shan, San Diego, CA (US)

Correspondence Address: FISH & RICHARDSON P.C., P.O. BOX 1022, MINNEAPOLIS, MN, 55440-1022, US

(21) Appl. No.: 11/992,806

(22) PCT Filed: Sep. 28, 2006
(86) PCT No.: PCT/US2006/038267

§ 371 Date: Sep. 19, 2008

Related U.S. Application Data
(60) Provisional application No. 60/721,783, filed on Sep. 29, 2005.

(51) Int. Cl. A61K 31/415; A61K 9/48

(52) U.S. Cl. 424/456; 514/406

(57) ABSTRACT

The present invention relates to certain pharmaceutical compositions of a 5-HT_{2A} serotonin receptor modulator and methods for preparing pharmaceutical composition related thereto. The pharmaceutical compositions are useful in the treatment of platelet aggregation, coronary artery disease, myocardial infarction, transient ischemic attack, angina, stroke, atrial fibrillation, reducing the risk of blood clot formation, asthma or symptoms thereof, agitation or a symptom, behavioral disorders, drug induced psychosis, excitative psychosis, Gilles de la Tourette's syndrome, manic disorder, organic or NOS psychosis, psychotic disorder, psychosis, acute schizophrenia, chronic schizophrenia, NOS schizophrenia and related disorders, sleep disorders, diabetic-related disorders, progressive multifocal leukoencephalopathy and the like.

This application is a 35 U.S.C. § 371 National Stage Application of International Appl. No. PCT/US2006/038267, filed Sep. 28, 2006, which claims the benefit of priority of U.S. Provisional Appl. No. 60/721,783, filed Sep. 29, 2005, each of which is hereby incorporated by reference in its entirety.

### FIELD OF THE INVENTION

The present invention relates to certain pharmaceutical compositions of a 5-HT_{2A} serotonin receptor modulator and methods for preparing pharmaceutical composition related thereto. The pharmaceutical compositions are useful in the treatment of platelet aggregation, coronary artery disease, myocardial infarction, transient ischemic attack, angina, stroke, atrial fibrillation, reducing the risk of blood clot formation, asthma or symptoms thereof, agitation or a symptom, behavioral disorders, drug induced psychosis, excitative psychosis, Gilles de la Tourette's syndrome, manic disorder, organic or NOS psychosis, psychotic disorder, psychosis, acute schizophrenia, chronic schizophrenia, NOS schizophrenia and related disorders, sleep disorders, diabetic-related disorders, progressive multifocal leukoencephalopathy and the like.

### BACKGROUND OF THE INVENTION

It has recently been discovered that certain 1,3-disubstituted urea compounds are modulators of the 5-HT_{2A} serotonin receptor and thus are useful for treating patients with disorders related thereto. Disorders related to the 5HT_{2A} serotonin receptor include, for example, platelet aggregation, coronary artery disease, myocardial infarction, transient ischemic attack, angina, stroke, atrial fibrillation, reducing the risk of blood clot formation, asthma or symptoms thereof, agitation or a symptom, behavioral disorders, drug induced psychosis, excitative psychosis, Gilles de la Tourette's syndrome, manic disorder, organic or NOS psychosis, psychotic disorder, psychosis, acute schizophrenia, chronic schizophrenia, NOS schizophrenia and related disorders, sleep disorders, diabetic-related disorders, progressive multifocal leukoencephalopathy, and the like.

The 1,3-disubstituted urea compounds are disclosed and claimed in International Application No. PCT/US2004/023488 (published as International Publication No. WO 2005/012254), incorporated herein by reference in its entirety, and can be prepared according to the procedures described therein.

In particular, the Compound of Formula (I), referred herein as 1-[3-(4-bromo-2-methyl-2H-pyrazol-3-yl)-4-methoxy-phenyl]-3-(2,4-difluoro-phenyl)-urea, has been found to be especially effective as a modulator of the 5-HT_{2A} serotonin receptor.

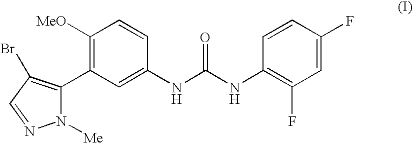

However, 1-[3-(4-bromo-2-methyl-2H-pyrazol-3-yl)-4-methoxy-phenyl]-3-(2,4-difluoro-phenyl)-urea was observed to have aqueous solubility of about 10 μg/mL or less in each of the following aqueous systems: (a) deionized water, (b) 0.01 N HCl (about pH 2), (c) phosphate buffer (about pH 7) and (d) saline (about 0.9% NaCl solution). Accordingly, 1-[3-(4-bromo-2-methyl-2H-pyrazol-3-yl)-4-methoxy-phenyl]-3-(2,4-difluoro-phenyl)-urea is considered to possess extremely poor aqueous solubility and would be expected to provide very low oral bioavailability. It is well known that an active drug substance administered by any route must possess some aqueous solubility for systemic absorption and therapeutic response. Compounds that have poor solubility often exhibit either incomplete or erratic absorption and thus produce a minimal response at a desired dosage.

Recognizing the problems, it has now been discovered that pharmaceutical compositions for 1-[3-(4-bromo-2-methyl-2H-pyrazol-3-yl)-4-methoxy-phenyl]-3-(2,4-difluoro-phenyl)-urea, which are disclosed herein, provide (a) substantial solubility, (b) pharmaceutical acceptability, (c) ease of processability during product manufacture, and (d) high oral bioavailability. In particular, it has been observed that certain compositions of the present invention allow for the preparation of pharmaceutical compositions containing 1-[3-(4-bromo-2-methyl-2H-pyrazol-3-yl)-4-methoxy-phenyl]-3-(2,4-difluoro-phenyl)-urea in exceedingly high concentrations, such as concentrations up to about 350 mg/mL, thus allowing for convenient oral administration while at the same time achieving improved pharmacokinetic parameters, such as at least two fold higher bioavailability, compared to the aqueous suspension.

Accordingly, the present invention relates to novel pharmaceutical compositions for 1-[3-(4-bromo-2-methyl-2H-pyrazol-3-yl)-4-methoxy-phenyl]-3-(2,4-difluoro-phenyl)-urea which are useful for treating 5HT_{2A} serotonin receptor disorders.

### SUMMARY OF THE INVENTION

One aspect of the present invention pertains to pharmaceutical compositions

comprising:

1-[3-(4-bromo-2-methyl-2H-pyrazol-3-yl)-4-methoxy-phenyl]-3-(2,4-difluoro-phenyl)-urea; and

at least one pharmaceutical excipient selected from a glycofurol, a poloxamer, a polyethylene glycol, a polyoxyethylene alkyl ether, a polyoxyethylene oil, a polyoxyethylene sorbitan fatty acid ester, an acyl polyoxyethylene, a polyglycolyzed glyceride, and a hydroxyacyl polyoxyethylene.

One aspect of the present invention pertains to methods for treating a 5HT_{2A} disorder, such as those disorders described herein, in an individual comprising administering to the individual in need thereof a therapeutically effective amount of a pharmaceutical composition as described herein.

In some embodiments, the individual is a mammal.

In some embodiments, the mammal is a human.

In some embodiments, the pharmaceutical composition is administered orally, nasally sublingually, buccally, transdermally, vaginally or rectally.

In some embodiments, the pharmaceutical composition is administered orally.

One aspect of the present invention pertains to pharmaceutical compositions as described herein, for use in a method of treatment of the human or animal body by therapy.

One aspect of the present invention pertains to pharmaceutical compositions as described herein, for use in a method of treatment of a 5HT_{2A} related disorder of the human or animal body by therapy.

One aspect of the present invention pertains to uses of a pharmaceutical composition as described herein, for the production of a medicament for use in the treatment of a 5HT_{2A} related disorder.

One aspect of the present invention pertains to methods for preparing pharmaceutical compositions comprising:

1-[3-(4-bromo-2-methyl-2H-pyrazol-3-yl)-4-methoxy-phenyl]-3-(2,4-difluoro-phenyl)-urea; and

a polyoxyethylene oil and a polyglycolyzed glyceride,

wherein the method comprises:

dissolving 1-[3-(4-bromo-2-methyl-2H-pyrazol-3-yl)-4-methoxy-phenyl]-3-(2,4-difluoro-phenyl)-urea into the polyoxyethylene oil or the polyglycolyzed glyceride, or a mixture thereof.

These and other aspects of the invention disclosed herein will be set forth in greater detail as the patent disclosure proceeds.

### DETAILED DESCRIPTION OF THE INVENTION

### Definitions

IN NEED OF TREATMENT as used herein refers to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL as used herein refers to any animal, including mammals, preferably mice, rats, other rodents, rabbits, dogs, cats, swine, cattle, sheep, horses, or primates, and most preferably humans.

INVERSE AGONISTS shall mean moieties that bind the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomotology of the disease,

(2) Inhibiting the disease; for example, inhibiting a disease, condition or disorder in an individual that is experiencing or displaying the pathology or symptomotology of the disease, condition or disorder (i.e., arresting further development of the pathology and/or symptomotology), and

(3) Ameliorating the disease; for example, ameliorating a disease, condition or disorder in an individual that is experiencing or displaying the pathology or symptomotology of the disease, condition or disorder (i.e., reversing the pathology and/or symptomotology).

The term “Glycofurol” as used herein refers to a class of pharmaceutical excipients comprising or consisting essentially of either a single compound or a mixture of compounds prepared from tetrahydrofurfuryl alcohol and ethylene oxide, or tetrahydrofurfuryl alcohol and a polyethylene glycol. In some embodiments, an excipient in this class comprises or consists essentially of a single compound or a mixture of compounds of the following formula:

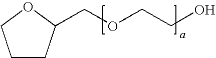

wherein “a” is an integer selected from 1 to 23. Representative examples, include but not limited to, Glycofurol® 75 (average molecular weight of about 190), and the like.

The term “Poloxamer” as used herein refers to a class of pharmaceutical excipients comprising or consisting essentially of either a single compound or a mixture of compounds prepared from synthetic block copolymers of ethylene oxide and propylene oxide. In some embodiments, an excipient in this class comprises or consists essentially of a single compound or a mixture of compounds of the following formula:

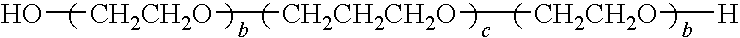

wherein “b” at each occurrence is independently an integer between 1 to 102; “c” is an integer between 1 and 57; b+c+b is 3 to 327; and the average molecule weight of the poloxamer is about 17500 or less. Poloxamers are known or can be prepared by methods in the art. A number of poloxamers are commercially available. Representative examples of a Poloxamer include, but are not limited to, Poloxamer 124 (Pluronic® L44NF), Poloxamer 188 (Pluronic® F68NF), Poloxamer 237 (Pluronic® F87NF), Poloxamer 338 (Pluronic® F108NF), Poloxamer 407 (Pluronic® F127NF), and the like.

The term “Polyethylene glycol” (also called macrogols) as used herein refers to a class of pharmaceutical excipients comprising or consisting essentially of either a single compound or a mixture of compounds of the following formula:

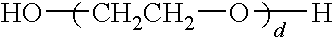

wherein “d” is an integer selected from 1 to 23. Polyethylene glycols are known or can be prepared by methods in the art. A number of polyethylene glycols are commercially available. Other polyethylene glycols can be prepared by synthetic methods available to those in the art. Representative examples of a polyethylene glycol include, but not limited to, PEG 200 (an average molecular weight of about 190-210), PEG 300 (an average molecular weight of about 285-315), PEG 400 (an average molecular weight of about 380420), PEG 540 (an average molecular weight of about 500-600), PEG 600 (an average molecular weight of about 570-613), PEG 900 (an average molecular weight of about 855-900), and the like.

It is understood in the art that the number that follows PEG indicates the average molecular weight of the polymer. For example, the term “PEG 300” refers to a PEG having an average molecular weight of 300, and in general, an average molecular weight range of about 285 to about 315. Unfortunately, another method to exists to describe PEG. Instead of using the average molecular weight, the number that follows “PEG” refers to the number of ethylene oxide units present, this number is expressed in whole numbers. Accordingly, using this nomenclature, PEG 6 would represent a PEG having an average number of ethylene oxide units of 6 and an average weight of 300. One skilled in the art appreciates and would understand both nomenclatures.

It is understood that instead of an average molecular weight PEG that an exact molecular weight PEG can also be used in the various embodiments of the present invention and accordingly, it is understood that where PEG is separate or part of a general description of an excipient described herein, that both an exact molecular weight PEG and an average molecular weight PEG are embraced by the present invention. Methods are known to those skilled in the art on how to prepare and identify PEGs of exact molecular weight and average molecular weight.

The term “Polyoxyethylene alkyl ether” as used herein refers to a class of pharmaceutical excipients comprising or consisting essentially of either a single compound or a mixture of compounds that can be generally described as a polyoxyethylene glycol monoethers of C₁₋₂₅ alcohols. In some embodiments, alcohols include, but not limited to, methyl-OH, ethyl-OH, propyl-OH, butyl-OH, pentyl-OH, hexyl-OH, lauryl-OH, myristyl-OH, cetyl-OH, stearyl-OH, and the like. In some embodiments, an excipient in this class comprises or consists essentially of a single compound or a mixture of compounds of the following formula:

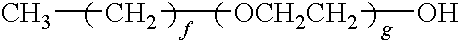

wherein “f” is an integer selected from 0 to 24, and “g” is an integer selected from 1 to 60. In some embodiments, “f” is selected from 1, 11, 13, 15, and 17 (i.e. together with the terminal methyl group to which the —(CH₂)ₜ is bonded forms ethyl, dodecyl, tetradecyl, hexadecyl and octadecyl groups respectively). In some embodiments, “f” is an integer selected from 1 to 24. In some embodiments the polyoxyethylene alkyl ethers is a mixture of different polymers. In general when a polyoxyethylene alkyl ether is described, the description refers to the major component of the excipient, such as the formula directly above, and in some examples other minor components can exist which are typically resulting from the preparation. Many polyoxyethylene alkyl ethers are known in the art with most being commercially available. Representative examples of a polyoxyethylene alkyl ether include, but are not limited to, diethylene glycol monoethyl ether (Transcutol P®), Cetomacrogol 1000, polyoxyl 2 cetyl ether, polyoxyl 10 cetyl ether, polyoxyl 20 cetyl ether, polyoxyl 4 lauryl ether, polyoxyl 9 lauryl ether, polyoxyl 23 lauryl ether, polyoxyl 2 oleyl ether, polyoxyl 10 oleyl ether, polyoxyl 20 oleyl ether, polyoxyl 2 stearyl ether, polyoxyl 10 stearyl ether, polyoxyl 20 stearyl ether, polyoxyl 100 stearyl ether, and the like.

The term “Polyoxyethylene sorbitan fatty acid esters” as used herein refers to a class of pharmaceutical excipients comprising or consisting essentially of either a single compound or a mixture of compounds prepared from sorbitol, anhydrides and ethylene oxide to give mono-, di-, tri- and/or tetra-fatty acid esters of polyoxyethylene sorbitan. In some embodiments, an excipient in this class comprises or consists essentially of a single compound or a mixture of compounds of the following formula:

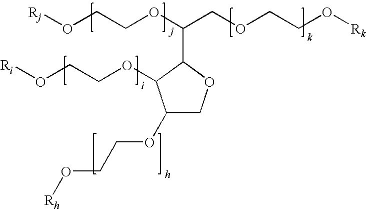

wherein “h”, “i”, “j”, and “k” are each independently an integer selected from 0 to 20; and Rₕ, Rᵢ, Rⱼ, and Rₖ are each independently H or an acyl group of a fatty acid; provided that “h+i+j+k” is an integer of 25 or less, including diastereomers, entiomers and mires thereof, and that at least one Rₕ, Rᵢ, Rⱼ, and Rₖ group is other than H. In one embodiment, the stereochemistry for the tetrahydro-furanyl ring is as follows:

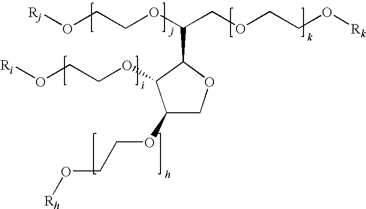

Examples of polyoxyethylene sorbitan fatty acid esters include, but not limited to, polysorbate 20 [polyoxyethylene 20 sorbitan monolaurate], polysorbate 21 [polyoxyethylene (4) sorbitan monolaurate], polysorbate 40 [polyoxyethylene 20 sorbitan monopalmitate], polysorbate 60 [polyoxyethylene 20 sorbitan monostearate], polysorbate 61 [polyoxyethylene (4) sorbitan monostearate], polysorbate 65 [polyoxyethylene 20 sorbitan tristearate], polysorbate 80 [polyoxyethylene 20 sorbitan monooleate], polysorbate 81 [polyoxyethylene (5) sorbitan monooleate], and the like. For polysorbates 20, 40, 60, 65, and 80, h+i+j+k=20; for polysorbate 81, b+i+j+k=5; and for polysorbates 21 and 61, h+i+j+k=4. Certain polysorbates are commercially available under the trade name “TWEEN” from Roche Applied Science.

The term “Acyl polyoxyethylene” as used herein refers to a class of pharmaceutical excipients comprising or consisting essentially of either a single compound or a mixture of compounds prepared from fatty acids and polyethylene glycol to give mono- and/or di-fatty esters of polyethylene glycol. In some embodiments, an excipient in this class comprises or consists essentially of a single compound or a mixture of compounds of the following formula:

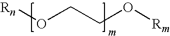

wherein “m” is an integer selected from 1 to 50; and Rₘ and Rₙ are each independently H or an acyl group of a fatty acid; provided that at least one Rₘ and Rₙ is a group other than H (i.e., both Rₘ and Rₙ are not H). In some embodiments, both Rₘ and Rₙ are independently an acyl group of a fatty acid. Examples of acyl polyoxyethylenes include, but not limited to, polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 4 distearate, polyoxyl 8 distearate, polyoxyl 12 distearate, polyoxyl 32 distearate, and the like.

The term “Hydroxyacyl polyoxyethylene” as used herein refers to a class of pharmaceutical excipients comprising or consisting essentially of either a single compound or a mixture of compounds of the following formula:

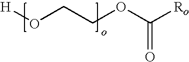

wherein “o” is an integer selected from 1 to 50; and Rₒ is a C₁₋₂₀ alkyl substituted with one or more —ORₚ group, wherein Rₚ is H or an acyl group of a fatty acid. When more than one —ORₚ group is present they can be the same or different. In some embodiments, Rₒ is a C₁₋₂₀ alkyl substituted with one —ORₚ group. Examples of hydroxyacyl polyoxyethylenes include, but not limited to, polyethylene glycol 660 12-hydroxystearate (polyethylene glycol-15-hydroxystearate, Solutol® HS 15), and the like.

The term “Polyglycolyzed glyceride” as used herein refers to a class of pharmaceutical excipients that is a combination of compounds comprising or consisting essentially of:

1) mono-, di-, and/or tri-esters of glycerol, and

2) mono- and/or di-esters of a polyethylene glycol of about 100 molecular weight to about 2,200 molecular weight with carboxylic acids, such as fatty acids.

In some embodiments, the polyglycolyzed glycerides contain minor amounts of glycerol and/or polyethylene glycols, for example, present in about 50% to about 0.001%, about 40% to about 0.001%, about 30% to about 0.001%, about 20% to about 0.001%, or about 10% to about 0.001% by weight of the total weight of the excipient either together or separately.

In some embodiments, the polyglycolyzed glyceride comprises or consists essentially of a combination of:

1) a single compound of Formula (II), or a mixture of compounds of Formula (II); and

2) a single compound of Formula (III), or a mixture of compounds of Formula (III);

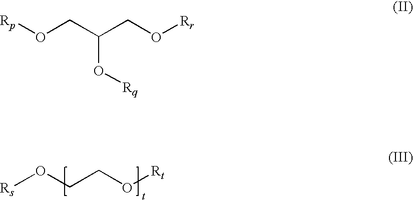

wherein “t” is an integer selected from 1 to 32; and Rₚ, R_{q}, Rᵣ, Rₛ, and Rₜ are each independently H or an acyl group of a fatty acid; including diastereomers, entiomers and mixtures thereof. In some embodiments, “t” is an integer selected from 4 to 32.

In general, polyglycolyzed glycerides are well defined mixtures and many are commercially available or prepared by methods known in the art. Examples of a polyglycolyzed glyceride include, but not limited to, (GELUCIRE® 35/10), (GELUCIRE® 44/14), (GELUCIRE® 46/07), PEG 1450 palmitostearic glycerides (PEG-32 glyceryl palmitostearate, GELUCIRE® 50/13), PEG 1450 stearic glycerides (PEG-32 glyceryl stearate, GELUCIRE® 53/10), PEG 8 caprylic/capric glycerides (PEG 400 caprylic/capric glycerides, LABRASOL®, Acconon® MC-8), PEG 300 caprylic/capric glycerides (SOFTIGEN® 767, Acconon® CC-6 EP), PEG 300 oleic glycerides (Labrafil® M 1944 CS), PEG 300 linoleic glycerides (Labrafil® M 2125 CS), PEG 1500 lauric glycerides (Acconon® C-44 EP), and the like.

The term “Polyoxyethylene oil” as used herein refers to a class of pharmaceutical excipients comprising or consisting essentially of either a single compound or a mixture of compounds obtained from the reaction of varying amounts of ethylene oxide with a mono-, di- and/or tri-substituted glyceride or mixtures thereof, wherein the substitution on the glyceride is a hydroxyl substituted fatty acid acyl group and when more than one hydroxyl substituted fatty acid acyl group is present then they can be the same or different. In some embodiments, the reaction of varying amounts of ethylene oxide is conducted with either caster oil or hydrogenated caster oil and they are referred herein as a “Polyoxyethylene caster oil” and a “Polyoxyethylene hydrogenated caster oil” respectively. In some embodiments, an excipient in this class comprises or consists essentially of a single compound or a mixture of compounds of the following formula:

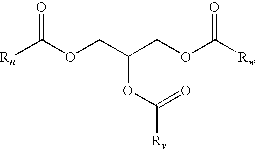

wherein Rᵤ, Rᵥ, and R_{w} are each independently H or a group selected from the Formula (IV) and Formula (V):

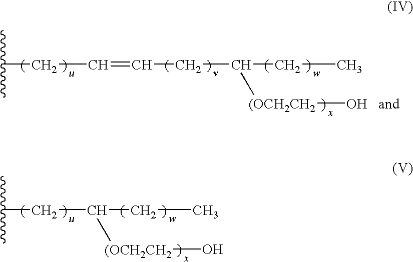

wherein “u”, “v”, and “w” are each independently an integer selected from 0 to 10 for each Rᵤ, Rᵥ, and R_{w}; and “x” is independently an integer selected from 1 to 45 for each Rᵤ, Rᵥ, and R_{w}, including diastereomers, entiomers, geometric isomers, and mixtures thereof. In some embodiments, Rᵤ, Rᵥ, and R_{w} are independently a group selected from the Formulae (IV) and (V) (i.e., a non-hydrogen group). In some embodiments, “u”, “v”, and “w” are the same for each Rᵤ, Rᵥ, and R_{w}. In some embodiments, at least one “u”, “v”, and “w” is different in Rᵤ, Rᵥ, and R_{w}. In some embodiments, “u” is 7, “v” is 0, and “w” is 6 and each Rᵤ, Rᵥ, and R_{w} are the same group. In general, a polyoxyethylene oil is a well defined mixture and many are commercially available. Examples of a polyoxyethylene oil include, but not limited to, polyoxyl 5 castor oil (Acconon CA-5; polyoxyethylene 5 castor oil), polyoxyl 9 castor oil (Acconon CA-9; polyoxyethylene 9 castor oil), polyoxyl 15 castor oil (Acconon CA-15; polyoxyethylene 15 castor oil), polyoxyl 35 castor oil (Cremophor™ EL; polyoxyethylene 35 castor oil), polyoxyl 40 castor oil (Castor oil POE-40; polyoxyethylene 40 castor oil), polyoxyl 40 hydrogenated castor oil (Cremophor™ RH 40; polyoxyethylene 40 hydrogenated castor oil), polyoxyl 60 hydrogenated castor oil (Eumulgin HRE 60; polyoxyethylene 60 hydrogenated castor oil), and the like. Examples of a polyoxyethylene hydrogenated castor oil include, but not limited to, polyoxyl 40 hydrogenated castor oil (Cremophor™ RH 40; polyoxyethylene 40 hydrogenated castor oil), polyoxyl 60 hydrogenated castor oil (Eumulgin HRE 60; polyoxyethylene 60 hydrogenated castor oil), and the like.

The term “fatty acid” as used herein refers to a carboxylic acid represented by the formula R₁C(═O)OH, wherein R₁ is a straight or branched C₃-C₂₅ alkyl group or a straight or branched C₃-C₂₅ alkenyl group, and R₁ is optionally substituted with 1, 2, 3, or 4 hydroxyl groups. It is understood that C₃-C₂₅ alkenyl can have one or more double-bond and each can be either cis or trans; when more than one double-bond is present they can be cis or traits or a mixture thereof.

In some embodiments, a fatty acid can be represented by the following formula: CH₃(CH₂)_{y}CH(R_{y})(CH₂)_{z}C(═O)OH wherein R_{y} is H or hydroxyl; and “y” and “z” are each independently an integer selected from 0 to 25, provided that y+z=23 or less. Accordingly, the acyl group of this fatty acid can be represented by the formula: CH₃(CH₂)_{y}CH(R_{y})(CH₂)_{z}C(═O)—.

In some embodiments, a fatty acid can be represented by the following formula: CH₃(CH₂)_{y}CH═CH(CH₂)_{z}C(═O)OH wherein “y” and “z” are each independently an integer selected from 0 to 25, provided that y+z=22 or less. In some embodiments, the double bond is cis. In some embodiments, the double bond is trans. Accordingly, the acyl group of this fatty acid can be represented by the formula: CH₃(CH₂)_{y}CH═CH(CH₂)_{z}C(═O)—.

Examples of saturated fatty acids include, but not limited to, propionic acid [CH₃CH₂C(═O)OH], butyric acid [CH₃(CH₂)₂C(═O)OH], pentanoic acid [CH₃(CH₂)₃C(═O)OH], hexanoic acid [CH₃(CH₂)₄C(═O)OH], heptanoic acid [CH₃(CH₂)₅C(═O)OH], caprylic acid [octanoic acid, CH₃(CH₂)₆C(═O)OH], nonanoic acid [pelargonic acid, CH₃(CH₂)₇C(═O)OH], capric acid [decanoic acid, CH₃(CH₂)₈C(═O)OH], undecanoic acid [CH₃(CH₂)₉C(═O)OH], lauric acid [dodecanoic acid, CH₃(CH₂)₁₀C(═O)OH], tridecanoic acid [CH₃(CH₂)₁₁C(═O)OH], myristic acid [tetradecanoic acid, CH₃(CH₂)₁₂C(═O)OH], palmitic acid [hexadecanoic acid, CH₃(CH₂)₁₄C(═O)OH], stearic acid [octadecanoic acid, CH₃(CH₂)₁₆C(═O)OH], 12-hydroxystearic acid [12-hydroxyoctadecanoic acid, CH₃(CH₂)₅CH(OH)(CH₂)₁₀C(═O)OH], arachidic acid [eicosanoic acid, CH₃(CH₂)₁₈C(═O)OH], and the like.

Examples of unsaturated fatty acids include, but not limited to, (alpha)-linolenic acid [CH₃CH₂CH═CHCH₂CH═CHCH₂CH═CH(CH₂)₇C(═O)OH], docosahexaenoic acid (commonly known as DHA; 22:6 omega-3), eicosapentaenoic acid (commonly known as EPA; C₂₀H₃₀O₂, all-cis-fatty acid 20:5 omega-3), linoleic acid [CH₃(CH₂)₄CH═CHCH₂CH═CH(CH₂)₇C(═O)OH], ricinoleic acid [castor oil acid; cis-12-hydroxyoctadec-9-enoic acid, CH₃(CH₂)₅CH(OH)CH₂CH═CH(CH₂)₇C(═O)OH], arachidonic acid [CH₃(CH₂)₄CH═CHCH₂CH═CHCH₂CH═CHCH₂CH═CH(CH₂)₃C(═O)OH], oleic acid [CH₃(CH₂)₇CH═CH(CH₂)₇C(═O)OH], erucic acid [CH₃(CH₂)₇CH═CH(CH₂)₁₁C(═O)OH], and the like.

The term “palmitostearic” refers to a mixture of palmitic acid and stearic acid. In some embodiments, the mixture of palmitic acid and stearic acid is a ratio of about 2:3 to about 3:2.

The phrase “acyl group of a fatty acid” as used herein refers to the R₁ group of a fatty acid bonded directly to the carbon of a carbonyl group and can be represented by the formula: R₁C(═O)—, wherein the R₁ has the same definition as described herein above.

The term “hydroxyl substituted fatty acid acyl group” as used herein refers to the R₁ group of a fatty acid bonded directly to the carbon of a carbonyl group and can be represented by the formula: R₁C(═O)— wherein R₁ is a straight or branched C₃-C₂₅ alkyl group or a straight or branched C₃-C₂₅ alkenyl group, and R₁ is substituted with 1, 2, 3, or 4 hydroxyl groups. It is understood that C₃-C₂₅ alkenyl can have one or more double-bond and each can be either cis or trans; when more than one double-bond is present they can be cis or Trans or a mixture thereof.

### Pharmaceutical Compositions of the Present Invention.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

One aspect of the present invention pertains to pharmaceutical compositions comprising: 1-[3-(4-bromo-2-methyl-2H-pyrazol-3-yl)-4-methoxy-phenyl]-3-(2,4-difluoro-phenyl)-urea; and

at least one pharmaceutical excipient selected from a glycofurol, a poloxamer, a polyethylene glycol, a polyoxyethylene alkyl ether, a polyoxyethylene oil, a polyoxyethylene sorbitan fatty acid ester, an acyl polyoxyethylene, a polyglycolyzed glyceride, and a hydroxyacyl polyoxyethylene.

In some embodiments, pharmaceuticals compositions of the present invention embrace solvates and hydrates of 1-[3-(4-bromo-2-methyl-2H-pyrazol-3-yl)-4-methoxy-phenyl]-3-(2,4-difluoro-phenyl)-urea.

In some embodiments, the at least one pharmaceutical excipient is selected from a polyethylene glycol, a polyoxyethylene alkyl ether, a polyoxyethylene oil, a polyoxyethylene sorbitan fatty acid ester, and a polyglycolyzed glyceride.

In some embodiments, the at least one pharmaceutical excipient is selected from diethylene glycol monoethyl ether, PEG 300, PEG 400, PEG 600, PEG 8 caprylic/capric glycerides, polyoxyl 40 hydrogenated castor oil, polysorbate 80, polysorbate 20, PEG 300 oleic glycerides, PEG 300 linoleic glycerides, and PEG 300 caprylic/capric glycerides.

In some embodiments, the present invention pertains to pharmaceutical compositions comprising:

a first pharmaceutical excipient that is a polyoxyethylene oil; and

a second pharmaceutical excipient selected from diethylene glycol monoethyl ether, PEG 300, PEG 400, PEG 600, PEG 8 caprylic/capric glycerides, polysorbate 80, polysorbate 20, PEG 300 oleic glycerides, PEG 300 linoleic glycerides, and PEG 300 caprylic/capric glycerides.

In some embodiments, the present invention pertains to pharmaceutical compositions comprising:

a first pharmaceutical excipient that is a polyglycolyzed glyceride; and

a second pharmaceutical excipient selected from diethylene glycol monoethyl ether, PEG 300, PEG 400, PEG 600, polyoxyl 40 hydrogenated castor oil, polysorbate 80, polysorbate 20, PEG 300 oleic glycerides, PEG 300 linoleic glycerides, and PEG 300 caprylic/capric glycerides.

In some embodiments, the polyoxyethylene oil is a polyoxyethylene hydrogenated castor oil.

In some embodiments, the polyoxyethylene hydrogenated castor oil is polyoxyl 40 hydrogenated castor oil.

In some embodiments, the polyglycolyzed glyceride is PEG 8 caprylic/capric glycerides.

In some embodiments, the present invention pertains to pharmaceutical compositions comprising: a polyoxyethylene oil and a polyglycolyzed glyceride.

In some embodiments, the present invention pertains to pharmaceutical compositions comprising: polyoxyl 40 hydrogenated castor oil and PEG 8 caprylic/capric glycerides.

In some embodiments, the present invention pertains to pharmaceutical compositions comprising: polyoxyl 40 hydrogenated castor oil and PEG 8 caprylic/capric glycerides together in an amount of at least about 60%, about 62%, about 64%, about 66%, about 68%, about 70%, about 72%, about 74%, about 76%, about 78%, about 80%, about 82%, about 84%, about 85%, about 86%, about 87%, about 88%, about 89%, about 90%, about 91%, about 92%, about 93%, about 94%, about 95%, about 96%, about 97%, about 98%, about 99%, or about 99.5% by weight of the total composition.

In some embodiments, the present invention pertains to pharmaceutical compositions comprising: polyoxyl 40 hydrogenated castor oil and PEG 8 caprylic/capric glycerides together in an amount of at least about 60% by weight of the total composition.

In some embodiments, the present invention pertains to pharmaceutical compositions comprising: polyoxyl 40 hydrogenated castor oil and PEG 8 caprylic/capric glycerides together in an amount of at least about 70% by weight of the total composition.

In some embodiments, the present invention pertains to pharmaceutical compositions comprising: polyoxyl 40 hydrogenated castor oil and PEG 8 caprylic/capric glycerides together in an amount of at least about 80% by weight of the total composition.

In some embodiments, the present invention pertains to pharmaceutical compositions comprising: polyoxyl 40 hydrogenated castor oil and PEG 8 caprylic/capric glycerides together in an amount of at least about 85% by weight of the total composition.

In some embodiments, the present invention pertains to pharmaceutical compositions comprising: polyoxyl 40 hydrogenated castor oil and PEG 8 caprylic/capric glycerides together in an amount of at least about 90% by weight of the total composition.

In some embodiments, the present invention pertains to pharmaceutical compositions comprising: polyoxyl 40 hydrogenated castor oil and PEG 8 caprylic/capric glycerides together in an amount of at least about 94% by weight of the total composition.

In some embodiments, the present invention pertains to pharmaceutical compositions comprising: polyoxyl 40 hydrogenated castor oil and PEG 8 caprylic/capric glycerides together in an amount of at least about 95% by weight of the total composition.

In some embodiments, the present invention pertains to pharmaceutical compositions comprising: polyoxyl 40 hydrogenated castor oil and PEG 8 caprylic/capric glycerides in a ratio of from about 1:99 to about 99:1, about 2:98 to about 98:2, about 3:97 to about 97:3, about 4:96 to about 96:4, about 5:95 to about 95:5, about 6:94 to about 94:6, about 7:93 to about 93:7, about 8:92 to about 92:8, about 9:91 to about 91:9, about 1:9 to about 9:1, about 11:89 to about 89:11, about 12:88 to about 88:12, about 13:87 to about 87:13, about 14:86 to about 86:14, about 15:85 to about 85:15, about 16:84 to about 84:16, about 17:83 to about 83:17, about 18:82 to about 82:18, about 19:81 to about 81:19, about 1:4 to about 4:1, about 21:79 to about 79:21, about 22:78 to about 78:22, about 23:77 to about 77:23, about 24:76 to about 76:24, about 25:75 to about 75:25, about 26:74 to about 74:26, about 27:73 to about 73:27, about 28:72 to about 72:28, about 29:71 to about 71:29, about 3:7 to about 7:3, about 31:69 to about 69:31, about 32:68 to about 68:32, about 33:67 to about 67:33, about 34:66 to about 66:34, about 35:65 to about 65:35, about 36:64 to about 64:36, about 37:63 to about 63:37, about 38:62 to about 62:38, about 39:61 to about 61:39, about 2:3 to about 3:2, about 41:59 to about 59:41, about 42:58 to about 58:42, about 43:57 to about 57:43, about 44:56 to about 56:44, about 45:55 to about 55:45, about 46:54 to about 54:46, about 47:53 to about 53:47, about 48:52 to about 52:48, or about 49:51 to about 51:49, each by weight.

In some embodiments, the present invention pertains to pharmaceutical compositions comprising: polyoxyl 40 hydrogenated castor oil and PEG 8 caprylic/capric glycerides in a ratio of from about 1:9 to about 9:1 by weight.

In some embodiments, the present invention pertains to pharmaceutical compositions comprising: polyoxyl 40 hydrogenated castor oil and PEG 8 caprylic/capric glycerides in a ratio of from about 2:3 to about 3:2 by weight.

In some embodiments, the present invention pertains to pharmaceutical compositions comprising: polyoxyl 40 hydrogenated castor oil and PEG 8 caprylic/capric glycerides in a ratio of from about 45:55 to about 55:45 by weight.

In some embodiments, the present invention pertains to pharmaceutical compositions comprising: polyoxyl 40 hydrogenated castor oil and PEG 8 caprylic/capric glycerides in a ratio of about 1:1 by weight.

In some embodiments, the 1-[3-(4-bromo-2-methyl-2H-pyrazol-3-yl)-4-methoxy-phenyl]-3-(2,4-difluoro-phenyl)-urea is in an amount of about 40% to about 0.00001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 40% to about 0.001%, about 39% to about 0.001%, about 38% to about 0.001%, about 37% to about 0.001%, about 36% to about 0.001%, about 35% to about 0.001%, about 34% to about 0.001%, about 33% to about 0.001%, about 32% to about 0.001%, about 31% to about 0.001%, about 30% to about 0.001%, about 29% to about 0.001%, about 28% to about 0.001%, about 27% to about 0.001%, about 26% to about 0.001%, about 25% to about 0.001%, about 24% to about 0.001%, about 23% to about 0.001%, about 22% to about 0.001%, about 21% to about 0.001%, about 20% to about 0.001%, about 19% to about 0.001%, about 18% to about 0.001%, about 17% to about 0.001%, about 16% to about 0.001%, about 15% to about 0.001%, about 14% to about 0.001%, about 13% to about 0.001%, about 12% to about 0.001%, about 11% to about 0.001%, about 10% to about 0.001%, about 9% to about 0.001%, about 8% to about 0.001%, about 7% to about 0.001%, about 6% to about 0.001%, about 5% to about 0.001%, about 4% to about 0.001%, about 3% to about 0.001%, about 2% to about 0.001%, or about 1% to about 0.001%, each by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 40% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 30% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 20% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 10% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 6% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 5% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 3% to about 0.001% by weight of the total composition.

In some embodiments, the 1-[3-(4-bromo-2-methyl-2H-pyrazol-3-yl)-4-methoxy-phenyl]-3-(2,4-difluoro-phenyl)-urea is in an amount of about 1% to about 0.001% by weight of the total composition.

In some embodiments, the present invention pertains to pharmaceutical compositions comprising:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 1:9 to about 9:1 by weight.

In some embodiments, the present invention pertains to pharmaceutical compositions comprising:

1-[3-(4-bromo-2-methyl-2H-pyrazol-3-yl)-4-methoxy-phenyl]-3-(2,4-difluoro-phenyl)-urea in an amount of about 400% to about 0.001% by weight of the total composition; and

a mixture of polyoxyl 40 hydrogenated castor oil and PEG 8 caprylic/capric glycerides in a ratio of about 2:3 to about 3:2 by weight.

In some embodiments, the present invention pertains to pharmaceutical compositions comprising:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 45:55 to about 55:45 by weight.

In some embodiments, the present invention pertains to pharmaceutical compositions comprising:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 1:1 by weight.

In some embodiments, the present invention pertains to pharmaceutical compositions consisting essentially of:

1-[3-(4-bromo-2-methyl-2H-pyrazol-3-yl)-4-methoxy-phenyl]-3-(2,4-difluoro-phenyl)urea in an amount of about 40% to about 0.001% by weight of the total composition; and

a mixture of polyoxyl 40 hydrogenated castor oil and PEG 8 caprylic/capric glycerides in a ratio of about 1:9 to about 9:1 by weight.

In some embodiments, the present invention pertains to pharmaceutical compositions consisting essentially of:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 2:3 to about 3:2 by weight.

In some embodiments, the present invention pertains to pharmaceutical compositions consisting essentially of:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 45:55 to about 55:45 by weight.

In some embodiments, the present invention pertains to pharmaceutical compositions consisting essentially of:

1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and

a mixture of polyoxyl 40 hydrogenated castor oil and PEG 8 caprylic/capric glycerides in a ratio of about 1:1 by weight.

In some embodiments, the present invention pertains to pharmaceutical compositions wherein:

1-[3-(4-bromo-2-methyl-2H-pyrazol-3-yl)-4-methoxy-phenyl]-3-(2,4-difluoro-phenyl)-urea is in an amount of about 30% to about 0.001% by weight of the total composition; and

the mixture of polyoxyl 40 hydrogenated castor oil and PEG 8 caprylic/capric glycerides is in an amount of at least about 70% by weight of the total composition.

In some embodiments, the present invention pertains to pharmaceutical compositions wherein:

1-[3-(4-bromo-2-methyl-2H-pyrazol-3-yl)-4-methoxy-phenyl]-3-(2,4-difluoro-phenyl)-urea is in an amount of about 20% to about 0.001% by weight of the total composition; and

the mixture of polyoxyl 40 hydrogenated castor oil and PEG 8 caprylic/capric glycerides is in an amount of at least about 80% by weight of the total composition.

In some embodiments, the present invention pertains to pharmaceutical compositions wherein:

1-[3-(4-bromo-2-methyl-2H-pyrazol-3-yl)-4-methoxy-phenyl]-3-(2,4-difluoro-phenyl)-urea is in an amount of about 10% to about 0.001% by weight of the total composition; and

the mixture of polyoxyl 40 hydrogenated castor oil and PEG 8 caprylic/capric glycerides is in an amount of at least about 90% by weight of the total composition.

In some embodiments, the present invention pertains to pharmaceutical compositions wherein:

1-[3-(4-bromo-2-methyl-2H-pyrazol-3-yl)-4-methoxy-phenyl]-3-(2,4-difluoro-phenyl)-urea is in an amount of about 6% to about 0.001% by weight of the total composition; and

the mixture of polyoxyl 40 hydrogenated castor oil and PEG 8 caprylic/capric glycerides is in an amount of at least about 94% by weight of the total composition.

In some embodiments, the present invention pertains to pharmaceutical compositions encapsulated in a capsule.

In some embodiments; the present invention pertains to pharmaceutical compositions encapsulated in a soft-gelatin capsule.

In some embodiments, the present invention pertains to pharmaceutical compositions encapsulated in a hard gelatin or non-gelatin capsule.

In some embodiments, the present invention pertains to pharmaceutical compositions suitable for oral administration.

In some embodiments, the present invention pertains to pharmaceutical compositions whereby the pharmaceutical composition provides an AUC of about 30 ng·hr/mL to about 1050 ng·hr/mL after oral administration.

In some embodiments, the present invention pertains to pharmaceutical compositions whereby the pharmaceutical composition provides an AUC of about 30 ng·hr/mL to about 660 ng·hr/mL after oral administration.

In some embodiments, the present invention pertains to pharmaceutical compositions whereby the pharmaceutical composition provides a Cₘₐₓ of about 10 ng/mL to about 245 ng/mL after oral administration.

In some embodiments, the present invention pertains to pharmaceutical compositions whereby the pharmaceutical composition provides a Cₘₐₓ of about 10 ng/mL to about 170 ng/mL after oral administration.

In some embodiments, the present invention pertains to pharmaceutical compositions whereby the pharmaceutical composition provides a tₘₐₓ of about 20 minutes to about 150 minutes after oral administration.

In some embodiments, the present invention pertains to pharmaceutical compositions whereby the pharmaceutical composition provides a tₘₐₓ of about 20 minutes to about 130 minutes after oral administration.

In some embodiments, the present invention pertains to pharmaceutical compositions wherein the 1-[3-(4-bromo-2-methyl-2H-pyrazol-3-yl)-4-methoxy-phenyl]-3-(2,4-difluoro-phenyl)-urea is in an amount of about 1 mg to about 160 mg.

In some embodiments, the present invention pertains to pharmaceutical compositions wherein the 1-[3-(4-bromo-2-methyl-2H-pyrazol-3-yl)-4-methoxy-phenyl]-3-(2,4-difluoro-phenyl)-urea is in an amount of about 5 mg, about 10 mg, about 20 mg or about 40 mg.

In some embodiments, the present invention pertains to pharmaceutical compositions encapsulated in a soft-gelatin capsule for oral administration consisting essentially of:

1-[3-(4-bromo-2-methyl-2H-pyrazol-3-yl)-4-methoxy-phenyl]-3-(2,4-difluoro-phenyl)-urea in an amount of about 6% to about 0.001% by weight of the total composition; and

a mixture of polyoxyl 40 hydrogenated castor oil and PEG 8 caprylic/capric glycerides in a ratio of about 1:1 by weight, wherein the mixture is in an amount of at least about 94% by weight of the total composition;

whereby the composition after oral administration of about 5 mg dose to about 40 mg dose of 1-[3-(4-bromo-2-methyl-2H-pyrazol-3-yl)-4-methoxy-phenyl]-3-(2,4-difluoro-phenyl)-urea provides an AUC of about 30 ng·hr/mL to about 660 ng·hr/mL, a Cₘₐₓ of about 10 ng/mL to about 245 ng/mL, or at of about 20 minutes to about 130 minutes after oral administration.

In some embodiments, the polyoxyl 40 hydrogenated castor oil is Cremophor® RH 40.

In some embodiments, the PEG 8 caprylic/capric glycerides is Labrasol®.

One aspect of the present invention pertains to dosage forms comprising:

a) about 0.001 mg to about 1000 mg of 1-[3-(4-bromo-2-methyl-2H-pyrazol-3-yl)-4-methoxy-phenyl]-3-(2,4-difluoro-phenyl)-urea; and

b) a polyoxyethylene oil, a polyglycolyzed glyceride, or a mixture thereof.

One aspect of the present invention pertains to dosage forms comprising:

a) about 0.5 mg to about 500 mg of 1-[3-(4-bromo-2-methyl-2H-pyrazol-3-yl)-4-methoxy-phenyl]-3-(2,4-difluoro-phenyl)-urea; and

b) a polyoxyethylene oil, a polyglycolyzed glyceride, or a mixture thereof.

One aspect of the present invention pertains to dosage forms comprising: 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg of 1-[3-(4-bromo-2-methyl-2H-pyrazol-3-yl)-4-methoxy-phenyl]-3-(2,4-difluoro-phenyl)-urea.

One aspect of the present invention pertains to dosage forms comprising: 1 mg, 5 mg, 10 mg, 20 mg, 30 mg, 40 mg, 80 mg, or 100 mg of 1-[3-(4-bromo-2-methyl-2H-pyrazol-3-yl)-4-methoxy-phenyl]-3-(2,4-difluoro-phenyl)-urea.

In some embodiments, the dosage form comprises polyoxyl 40 hydrogenated castor oil and PEG 8 caprylic/capric glycerides.

In some embodiments, the dosage form comprises polyoxyl 40 hydrogenated castor oil and PEG 8 caprylic/capric glycerides in a ratio of from about 1:9 to about 9:1 by weight.

In some embodiments, the dosage form comprises polyoxyl 40 hydrogenated castor oil and PEG 8 caprylic/capric glycerides in a ratio of from about 2:3 to about 3:2 by weight.

In some embodiments, the dosage form comprises polyoxyl 40 hydrogenated castor oil and PEG 8 caprylic/capric glycerides in a ratio of from about 45:55 to about 55:45 by weight.

In some embodiments, the dosage form comprises polyoxyl 40 hydrogenated castor oil and PEG 8 caprylic/capric glycerides in a ratio of about 1:1 by weight.

In some embodiments, the dosage form is in solid form.

In some embodiments, the dosage form is in the form of a capsule.

In some embodiments, the dosage form is in the form of a soft-gel capsule.

In some embodiments, the dosage form is suitable for oral administration.

In some embodiments, the present invention pertains to pharmaceutical compositions wherein the polyoxyl 40 hydrogenated castor oil is Cremophor® RH 40.

In some embodiments, the present invention pertains to pharmaceutical compositions wherein the PEG 8 caprylic/capric glycerides is Labrasol®.

One aspect of the present invention pertains to methods for preparing a pharmaceutical composition comprising:

1-[3-(4-bromo-2-methyl-2H-pyrazol-3-yl)-4-methoxy-phenyl]-3-(2,4-difluoro-phenyl)-urea; and

a polyoxyethylene oil and a polyglycolyzed glyceride,

wherein the method comprises:

dissolving 1-[3-(4 bromo-2-methyl-2H-pyrazol-3-yl)-4-methoxy-phenyl]-3-(2,4-difluoro-phenyl)-urea into the polyoxyethylene oil or the polyglycolyzed glyceride, or a mixture thereof.

In some embodiments, the present invention pertains to methods of preparing pharmaceutical compositions wherein the dissolving is in the polyoxyethylene oil and the polyglycolyzed glyceride in a ratio of about 1:1 by weight.

In some embodiments, the present invention pertains to methods of preparing pharmaceutical compositions wherein the polyoxyethylene oil is polyoxyl 40 hydrogenated castor oil.

In some embodiments, the present invention pertains to methods of preparing pharmaceutical compositions wherein the polyglycolyzed glyceride is PEG 8 caprylic/capric glycerides.

In some embodiments, the present invention pertains to methods of preparing pharmaceutical compositions wherein the 1-[3-(4-bromo-2-methyl-2H-pyrazol-3-yl)-4-methoxy-phenyl]-3-(2,4-difluoro-phenyl)-urea is in an amount of about 40% to about 0.001% by weight of the total composition.

In some embodiments, the present invention pertains to methods of preparing pharmaceutical compositions wherein the 1-[3-(4-bromo-2-methyl-2H-pyrazol-3-yl)-4-methoxy-phenyl]-3-(2,4-difluoro-phenyl)-urea is in an amount of about 6% to about 0.001% by weight of the total composition.

In some embodiments, the present invention pertains to methods of preparing pharmaceutical compositions wherein the mixture is in an amount of at least about 60% by weight of the total composition.

In some embodiments, the present invention pertains to methods of preparing pharmaceutical compositions wherein the mixture is in an amount of at least about 94% by weight of the total composition.

In some embodiments, the present invention pertains to methods of preparing pharmaceutical compositions wherein the dissolving of 1-[3-(4-bromo-2-methyl-2H-pyrazol-3-yl)-4-methoxy-phenyl]-3-(2,4-difluoro-phenyl)-urea is at a temperature above about 25° C.

In some embodiments, the present invention pertains to methods of preparing pharmaceutical compositions wherein the dissolving of 1-[3-(4-bromo-2-methyl-2H-pyrazol-3-yl)-4-methoxy-phenyl]-3-(2,4-difluoro-phenyl)-urea is at a temperature in the range of about 25° C. to about 80° C.

In some embodiments, the present invention pertains to methods of preparing pharmaceutical compositions wherein the dissolving of 1-[3-(4-bromo-2-methyl-2H-pyrazol-3-yl)-4-methoxy-phenyl]-3-(2,4-fluoro-phenyl)-urea is at a temperature in the range of about 40° C. to about 70° C.

In some embodiments, the present invention pertains to methods of preparing pharmaceutical compositions wherein the dissolving of 1-[3-(4-bromo-2-methyl-2H-pyrazol-3-yl)-4-methoxy-phenyl]-3-(2,4-difluoro-phenyl)-urea is at a temperature in the range of about 55° C. to about 65° C.

In some embodiments, the present invention pertains to methods of preparing pharmaceutical compositions further comprising the step of cooling the pharmaceutical composition to about 25° C.

In some embodiments, the present invention pertains to methods of preparing pharmaceutical compositions further comprising the step of filling the pharmaceutical composition in a capsule.

In some embodiments, the present invention pertains to methods of preparing pharmaceutical compositions further comprising the step of filling the pharmaceutical composition in a soft gelatin capsule.

In some embodiments, the present invention pertains to methods of preparing pharmaceutical compositions further comprising the step of filling the pharmaceutical composition in a hard gelatin or non-gelatin capsule.

In some embodiments, the present invention pertains to methods of preparing pharmaceutical compositions wherein the polyoxyl 40 hydrogenated castor oil is Cremophor® RH 40.

In some embodiments, the present invention pertains to methods of preparing pharmaceutical compositions wherein the PEG 8 caprylic/capric glycerides is Labrasol®.

### Indications and Methods of Treatment

In addition to the foregoing beneficial uses, the pharmaceutical compositions of the present invention are useful in the treatment of several additional diseases and disorders, and in the amelioration of symptoms thereof. Without limitation, these include the following:

1. Antiplatelet Therapies (5HT_{2A} Mediated Platelet Aggregation):

Antiplatelet agents (antiplatelets) are prescribed for a variety of conditions. For example, in coronary artery disease they are used to help prevent myocardial infarction or stroke in patients who are at risk of developing obstructive blood clots (e.g., coronary thrombosis).

In a myocardial infarction (heart attack), the heart muscle does not receive enough oxygen-rich blood as a result of a blockage in the coronary blood vessels. If taken while an attack is in progress or immediately afterward (preferably within 30 minutes), antiplatelets can reduce the damage to the heart.

A transient ischemic attack (“TIA” or “mini-stroke”) is a brief interruption of oxygen flow to the brain due to decreased blood flow through arteries, usually due to an obstructing blood clot. Antiplatelet drugs have been found to be effective in preventing TIAs.

Angina is a temporary and often recurring chest pain, pressure or discomfort caused by inadequate oxygen-rich blood flow (ischemia) to some parts of the heart. In patients with angina, antiplatelet therapy can reduce the effects of angina and the risk of myocardial infarction.

Stroke is an event in which the brain does not receive enough oxygen-rich blood, usually due to blockage of a cerebral blood vessel by a blood clot. In high-risk patients, taking antiplatelets regularly has been found to prevent the formation blood clots that cause first or second strokes.

Angioplasty is a catheter based technique used to open arteries obstructed by a blood clot. Whether or not stenting is performed immediately after this procedure to keep the artery open, antiplatelets can reduce the risk of forming additional blood clots following the procedure(s).

Coronary bypass surgery is a surgical procedure in which an artery or vein is taken from elsewhere in the body and grafted to a blocked coronary artery, rerouting blood around the blockage and through the newly attached vessel. After the procedure, antiplatelets can reduce the risk of secondary blood clots.

Atrial fibrillation is the most common type of sustained irregular heart rhythm (arrythmia). Atrial fibrillation affects about two million Americans every year. In atrial fibrillation, the atria (the heart's upper chambers) rapidly fire electrical signals that cause them to quiver rather than contract normally. The result is an abnormally fast and highly irregular heartbeat. When given after an episode of atrial fibrillation, antiplatelets can reduce the risk of blood clots forming in the heart and traveling to the brain (embolism).

5HT_{2A} receptors are expressed on smooth muscle of blood vessels and 5-HT secreted by activated platelets causes vasoconstriction as well as activation of additional platelets during clotting. There is evidence that a 5HT_{2A} inverse agonist will inhibit platelet aggregation and thus be a potential treatment as an antiplatelet therapy (see Satimura, K, et al., Clin Cardiol 2002 Jan. 25 (1):28-32; and Wilson, H. C et al., Thromb Haemost 1991 Sep. 2; 66(3):355-60).

The 5HT_{2A} inverse agonist disclosed herein can provide beneficial improvement in microcirculation to patients in need of antiplatelet therapy by antagonizing the vasoconstrictive products of the aggregating platelets in, for example and not limited to the indications described above. Accordingly, in some embodiments, the present invention provides methods for reducing platelet aggregation in a patient in need thereof comprising administering to the patient a composition comprising the 5HT_{2A} inverse agonist disclosed herein. In further embodiments, the present invention provides methods for treating coronary artery disease, myocardial infarction, transient ischemic attack, angina, stroke, atrial fibrillation, or a symptom of any of the foregoing in a patient in need of the treatment, comprising administering to the patient a composition comprising the 5-HT_{2A} inverse agonist disclosed herein.

In further embodiments, the present invention provides methods for reducing risk of blood clot formation in an angioplasty or coronary bypass surgery patient, or a patient suffering from atrial fibrillation, comprising administering to the patient a composition comprising the 5HT_{2A} inverse agonist disclosed herein at a time where such risk exists.

2. Asthma

It has been suggested that 5-HT (5-hydroxytyptamine) plays a role in the pathophysiology of acute asthma (see Cazzola, M. and Matera, M. G., TFPS, 2000, 21, 13; and De Bie, J. J. et al., British J. Pharm, 1998, 124, 857-864). The pharmaceutical compositions of the present invention disclosed herein are useful in the treatment of asthma, and the treatment of the symptoms thereof. Accordingly, in some embodiments, the present invention provides methods for treating asthma in a patient in need of the treatment, comprising administering to the patient a composition comprising the 5HT_{2A} inverse agonist disclosed herein. In further embodiments, methods are provided for treating a symptom of asthma in a patient in need of the treatment, comprising administering to the patient a composition comprising the 5-HT_{2A} inverse agonist disclosed herein.

3. Agitation

Agitation is a well-recognized behavioral syndrome with a range of symptoms, including hostility, extreme excitement, poor impulse control, tension and uncooperativeness (See Cohen-Mansfield J, and Billig, N., (1986), Agitated Behaviors in the Elderly. I. A Conceptual Review. J Am Geriatr Soc 34(10): 711-721).

Agitation is a common occurrence in the elderly and often associated with dementia such as those caused by Alzheimer's disease, Lewy Body, Parkinson's, and Huntington's, which are degenerative diseases of the nervous system and by diseases that affect blood vessels, such as stroke, or multi-infarct dementia, which is caused by multiple strokes in the brain can also induce dementia. Alzheimer's disease accounts for approximately 50 to 70% of all dementias (See Koss E, et al., (1997), Assessing patterns of agitation in Alzheimer's disease patients with the Cohen-Mansfield Agitation Inventory. The Alzheimer's Disease Cooperative Study. Alzheimer Dis Assoc Disord 11(suppl 2):S45-S50).

An estimated five percent of people aged 65 and older and up to 20 percent of those aged 80 and older are affected by dementia; of these sufferers, nearly-half exhibit behavioral disturbances, such as agitation, wandering and violent outbursts.

Agitated behaviors can also be manifested in cognitively intact elderly people and by those with psychiatric disorders other than dementia.

Agitation is often treated with antipsychotic medications such as haloperidol in nursing home and other assisted care settings. There is emerging evidence that agents acting at the 5HT_{2A} receptors in the brain have the effects of reducing agitation in patients, including Alzeimer's dementia (See Katz, I. R., et al., J Clin Psychiatry 1999 Feb., 60(2):107-115; and Street, J. S., et al., Arch Gen Psychiatry 2000 Oct., 57(10):968-976).

The compositions of the invention disclosed herein are useful for treating agitation and symptoms thereof. Thus, in some embodiments, the present invention provides methods for treating agitation in a patient in need of such treatment comprising administering to the patient a composition comprising the 5-HT_{2A} inverse agonist disclosed herein. In some embodiments, the agitation is due to a psychiatric disorder other than dementia. In some embodiments, the present invention provides methods for treatment of agitation or a symptom thereof in a patient suffering from dementia comprising administering to the patient a composition comprising the 5-HT_{2A} inverse agonist disclosed herein. In some embodiments of such methods, the dementia is due to a degenerative disease of the nervous system, for example and without limitation, Alzheimers disease, Lewy Body, Parkinson's disease, and Huntington's disease, or dementia due to diseases that affect blood vessels, including, without limitation, stroke and multi-infarct dementia. In some embodiments, methods are provided for treating agitation or a symptom thereof in a patient in need of such treatment, where the patient is a cognitively intact elderly patient, comprising administering to the patient a composition comprising the 5HT_{2A} inverse agonist disclosed herein.

4. Add-On Therapy to Haloperidol in the Treatment of Schizophrenia and Other Disorders:

Schizophrenia is a psychopathic disorder of unknown origin, which usually appears for the first time in early adulthood and is marked by a number of characteristics, psychotic symptoms, progression, phasic development and deterioration in social behavior and professional capability in the region below the highest level ever attained. Characteristic psychotic symptoms are disorders of thought content (multiple, fragmentary, incoherent, implausible or simply delusional contents or ideas of persecution) and of mentality (loss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook).

Haloperidol (Haldol) is a potent dopamine D₂ receptor antagonist. It is widely prescribed for acute schizophrenic symptoms, and is very effective for the positive symptoms of schizophrenia. However, Haldol is not effective for the negative symptoms of schizophrenia and may actually induce negative symptoms as well as cognitive dysfunction. In accordance with some methods of the invention, adding the 5-HT_{2A} inverse agonist concomitantly with Haldol can provide benefits including the ability to use a lower dose of Haldol without losing its effects on positive symptoms, while reducing or eliminating its inductive effects on negative symptoms, and prolonging relapse to the patient's next schizophrenic event.

Haloperidol is used for treatment of a variety of behavioral disorders, drug induced psychosis, excitative psychosis, Gilles de la Tourette's syndrome, manic disorders, psychosis (organic and NOS), psychotic disorder, psychosis, schizophrenia (acute, chronic and NOS). Further uses include in the treatment of infantile autism, huntington's chorea, and nausea and vomiting from chemotherapy and chemotherapeutic antibodies. Administration of the 5-HT_{2A} inverse agonist disclosed herein with haloperidol also can provide benefits in these indications.

In some embodiments, the present invention provides methods for treating a behavioral disorder, drug induced psychosis, excitative psychosis, Gilles de la Tourette's syndrome, manic disorders, psychosis (organic and NOS), psychotic disorder, psychosis, schizophrenia (acute, chronic and NOS) comprising administering to the patient a dopamine D₂ receptor antagonist and the 5HT_{2A} inverse agonist disclosed herein.

In some embodiments, the present invention provides methods for treating a behavioral disorder, drug induced psychosis, excitative psychosis, Gilles de la Tourette's syndrome, manic disorders, psychosis (organic and NOS), psychotic disorder, psychosis, schizophrenia (acute, chronic and NOS) comprising administering to the patient haloperidol and the 5HT_{2A} inverse agonist disclosed herein.

In some embodiments, the present invention provides methods for treating infantile autism, huntington's chorea, or nausea and vomiting from chemotherapy or chemotherapeutic antibodies comprising administering to the patient a dopamine D₂ receptor antagonist and the 5HT_{2A} inverse agonist disclosed herein.

In some embodiments, the present invention provides methods for treating infantile autism, huntington's chorea, or nausea and vomiting from chemotherapy or chemotherapeutic antibodies comprising administering to the patient haloperidol and the 5HT_{2A} inverse agonist disclosed herein.

In further embodiments, the present invention provides methods for treating schizophrenia in a patient in need of the treatment comprising administering to the patient a dopamine D₂ receptor antagonist and the 5-HT_{2A} inverse agonist disclosed herein. Preferably, the dopamine D₂ receptor antagonist is haloperidol.

The administration of the dopamine D₂ receptor antagonist can be concomitant with administration of the 5HT_{2A} inverse agonist, or they can be administered at different times. Those of skill in the art will easily be able to determine appropriate dosing regimes for the most efficacious reduction or elimination of deleterions haloperidol effects. In some embodiments, haloperidol and the 5HT_{2A} inverse agonist are administered in a single dosage form, and in other embodiments, they are administered in separate dosage forms.

The present invention further provides methods of alleviating negative symptoms of schizophrenia induced by the administration of haloperidol to a patient suffering from the schizophrenia, comprising administering to the patient the 5-HT_{2A} inverse agonist as disclosed herein.

5. Sleep Disorders

It is reported in the National Sleep Foundation's 2002 Sleep In America Poll, more than one-half of the adults surveyed (58%) report having experienced one or more symptoms of insomnia at least a few nights a week in the past year. Additionally, about three in ten (35%) say they have experienced insomnia-like symptoms every night or almost every night.

The normal sleep cycle and sleep architecture can be disrupted by a variety of organic causes as well as environmental influences. According to the International Classification of Sleep Disorders, there are over 80 recognized sleep disorders. Of these, compounds of the present invention are effective, for example, in any one or more of the following sleep disorders (ICSD—International Classification of Sleep Disorders: Diagnostic and Coding Manual. Diagnostic Classification Steering Committee, American Sleep Disorders Association, 1990):

### A. Dyssomnias

a. Intrinsic Sleep Disorders:

Psychophysiological insomnia, Sleep state misperception, Idiopathic insomnia, Obstructive sleep apnea syndrome, Central sleep apnea syndrome, Central alveolar hypoventilation syndrome, Periodic limb movement disorder, Restless leg syndrome and Intrinsic sleep disorder NOS.

b. Extrinsic Sleep Disorders:

Inadequate sleep hygiene, Environmental sleep disorder, Altitude insomnia, Adjustment sleep disorder, Insufficient sleep syndrome, Limit-setting sleep disorder, SleepOnset association disorder, Nocturnal eating (drinking) syndrome, Hypnotic dependent sleep disorder, Stimulant-dependent sleep disorder, Alcohol-dependent sleep disorder, Toxin-induced sleep disorder and Extrinsic sleep disorder NOS.

c. Circadian Rhythm Sleep Disorders:

Time zone change (jet lag) syndrome, Shift work sleep disorder, Irregular sleep-wake pattern, Delayed sleep phase syndrome, Advanced sleep phase syndrome, Non-24 hour sleep-wake disorder and Circadian rhythm sleep disorder NOS.

### B. Parasomnias

a. Arousal Disorders:

Confusional arousals, Sleepwalking and Sleep terrors.

b. Sleep-Wake Transition Disorders:

Rhythmic movement disorder, Sleep starts, Sleep talking and Nocturnal leg cramps.

C. Sleep Disorders Associated with Medical/Psychiatric Disorders

a. Associated with Mental Disorders:

Psychoses, Mood disorders, Anxiety disorders, Panic disorders and Alcoholism.

b. Associated with Neurological Disorders:

Cerebral degenerative disorders, Dementia, Parkinsonism, Fatal familial insomnia, Sleep-related epilepsy, Electrical status epilepticus of sleep and Sleep-related headaches.

c. Associated with Other Medical Disorders:

Sleeping sickness, Nocturnal cardiac ischemia, Chronic obstructive pulmonary disease, Sleep-related asthma, Sleep-related gastroesophageal reflux, Peptic ulcer disease, Fibrositis syndrome, Osteoarthritis, Rheumatoid arthritis, Fibromyalgia and Post-surgical.

The effects of sleep deprivation are more than excessive daytime sleepiness. Chronic insomniacs report elevated levels of stress, anxiety, depression and medical illnesses (National Institutes of Health, National Heart, Lung, and Blood Institute, Insomnia Facts Sheet, October 1995). Preliminary evidence suggests that having a sleep disorder that causes significant loss of sleep may contribute to increased susceptibility to infections due to immunosuppression, cardiovascular complications such as hypertension, cardiac arrhythmias, stroke, and myocardial infarction, compromised glucose tolerance, increased obesity and metabolic syndrome. Compounds of the present invention are useful to prevent or alleviate these complications by improving sleep quality.

The most common class of medications for the majority of sleep disorders are the benzodiazepines, but the adverse effect profile of benzodiazepines include daytime sedation, diminished motor coordination, and cognitive impairments. Furthermore, the National Institutes of Health Consensus conference on Sleeping Pills and Insomnia in 1984 have developed guidelines discouraging the use of such sedative-hypnotics beyond 4-6 weeks because of concerns raised over drug misuse, dependency, withdrawal and rebound insomnia. Therefore, it is desirable to have a pharmacological agent for the treatment of insomnia, which is more effective and/or has fewer side effects than those currently used. In addition, benzodiazepines are used to induce sleep, but have little to no effect on the maintenance of sleep, sleep consolidation or slow wave sleep. Therefore, sleep maintenance disorders are not currently well treated.

Clinical studies with agents of a similar mechanism of action as is the compound disclosed herein have demonstrated significant improvements on objective and subjective sleep parameters in normal, healthy volunteers as well as patients with sleep disorders and mood disorders [Sharpley A L, et al. Slow Wave Sleep in Humans: Role of 5HT_{2A} and 5HT_{2C} Receptors. Neuropharmacology, 1994, Vol. 33(3/4):467-71; Winokur A, et al. Acute Effects of Mirtazapine on Sleep Continuity and Sleep Architecture in Depressed Patients: A Pilot Study. Soc of Biol Psych, 2000, Vol. 48:75-78; and Landolt H P, et al. Serotonin-2 Receptors and Human Sleep: Effect of Selective Antagonist on EEG Power Spectra. Neuropsychopharmacology, 1999, Vol. 21(3):455-66].

Some sleep disorders are sometimes found in conjunction with other conditions and accordingly those conditions are treatable by pharmaceutical compositions of the present invention. For example, but not limited to, patients suffering from mood disorders typically suffer from a sleep disorder that can be treated by pharmaceutical compositions of the present invention. Having one pharmacological agent which treats two or more existing or potential conditions, as does the present invention, is more cost effective, leads to better compliance and has fewer side effects than taking two or more agents.

It is an object of the present invention to provide a therapeutic agent for the use in treating Sleep Disorders. It is another object of the present invention to provide one pharmaceutical agent, which may be useful in treating two or more conditions wherein one of the conditions is a sleep disorder. Compositions of the present invention as described herein may be used alone or in combination with a mild sleep inducer (i.e. antihistamine).

### Sleep Architecture:

Sleep comprises two physiological states: Non rapid eye movement (NREM) and rapid eye movement (REM) sleep. NREM sleep consists of four stages, each of which is characterized by progressively slower brain wave patterns, with the slower patterns indicating deeper sleep. So called delta sleep, stages 3 and 4 of NREM sleep, is the deepest and most refreshing type of sleep. Many patients with sleep disorders are unable to adequately achieve the restorative sleep of stages 3 and 4. In clinical terms, patients' sleep patterns are described as fragmented, meaning the patient spends a lot of time alternating between stages 1 and 2 (semi-wakefulness) and being awake and very little time in deep sleep. As used herein, the term “fragmented sleep architecture” means an individual, such as a sleep disorder patient, spends the majority of their sleep time in NREM sleep stages 1 and 2, lighter periods of sleep from which the individual can be easily aroused to a Waling state by limited external stimuli. As a result, the individual cycles through frequent bouts of light sleep interrupted by frequent awakenings throughout the sleep period. Many sleep disorders are characterized by a fragmented sleep architecture. For example, many elderly patients with sleep complaints have difficulty achieving long bouts of deep refreshing sleep (NREM stages 3 and 4) and instead spend the majority of their sleep time in NREM sleep stages 1 and 2.

In contrast to fragmented sleep architecture, as used herein the term “sleep consolidation” means a state in which the number of NREM sleep bouts, particularly Stages 3 and 4, and the length of those sleep bouts are increased, while the number and length of waking bouts are decreased. In essence, the architecture of the sleep disorder patient is consolidated to a sleeping state with increased periods of sleep and fewer awakenings during the night and more time is spent in slow wave sleep (Stages 3 and 4) with fewer oscillation Stage 1 and 2 sleep. Compositions of the present invention can be effective in consolidating sleep patterns so that the patient with previously fragmented sleep can now achieve restorative, delta-wave sleep for longer, more consistent periods of time.

As sleep moves from stage 1 into later stages, heart rate and blood pressure drop, metabolic rate and glucose consumption fall, and muscles relax. In normal sleep architecture, NREM sleep makes up about 75% of total sleep time; stage 1 accounting for 5-10% of total sleep time, stage 2 for about 45-50%, stage 3 approximately 12%, and stage 4 13-15%. About 90 minutes after sleep onset, NREM sleep gives way to the first REM sleep episode of the night. REM makes up approximately 25% of total sleep time. In contrast to NREM sleep, REM sleep is characterized by high pulse, respiration, and blood pressure, as well as other physiological patterns similar to those seen in the active waking stage. Hence, REM sleep is also known as “paradoxical sleep.” Sleep onset occurs during NREM sleep and takes 10-20 minutes in healthy young adults. The four stages of NREM sleep together with a REM phase form one complete sleep cycle that is repeated throughout the duration of sleep, usually four or five times. The cyclical nature of sleep is regular and reliable; a REM period occurs about every 90 minutes during the night. However, the first REM period tends to be the shortest, often lasting less than 10 minutes, whereas the later REM periods may last up to 40 minutes. With aging, the time between retiring and sleep onset increases and the total amount of night-time sleep decreases because of changes in sleep architecture that impair sleep maintenance as well as sleep quality. Both NREM (particularly stages 3 and 4) and REM sleep are reduced. However, stage 1 NREM sleep, which is the lightest sleep, increases with age.

As used herein, the term “delta power” means a measure of the duration of EEG activity in the 0.5 to 3.5 Hz range during NREM sleep and is thought to be a measure of deeper, more refreshing sleep. Delta power is hypothesized to be a measure of a theoretical process called Process S and is thought to be inversely related to the amount of sleep an individual experiences during a given sleep period. Sleep is controlled by homeostatic mechanisms; therefore, the less one sleeps the greater the drive to sleep. It is believed that Process S builds throughout the wake period and is discharged most efficiently during delta power sleep. Delta power is a measure of the magnitude of Process S prior to the sleep period. The longer one stays awake, the greater Process S or drive to sleep and thus the greater the delta power during NREM sleep. However, individuals with sleep disorders have difficulty achieving and maintaining delta wave sleep, and thus have a large build-up of Process S with limited ability to discharge this buildup during sleep. 5-HT_{2A} agonists tested preclinically and clinically mimic the effect of sleep deprivation on delta power, suggesting that subjects with sleep disorders treated with a 5-HT_{2A} inverse agonist or antagonist will be able to achieve deeper more refreshing sleep. These same effects have not been observed with currently marketed pharmacotherapies. In addition, currently marketed pharmacotherapies for sleep have side effects such as hangover effects or addiction that are associated with the GABA receptor. 5HT_{2A} inverse agonists do not target the GABA receptor and so these side effects are not a concern.

### Subjective and Objective Determinations of Sleep Disorders:

There are a number of ways to determine whether the onset, duration or quality of sleep (e.g. non-restorative or restorative sleep) is impaired or improved. One method is a subjective determination of the patient, e.g., do they feel drowsy or rested upon waking. Other methods involve the observation of the patient by another during sleep, e.g., how long it takes the patient to fall asleep, how many times does the patient wake up during the night, bow restless is the patient during sleep, etc. Another method is to objectively measure the stages of sleep using polysomnography.

Polysomnography is the monitoring of multiple electrophysiological parameters during sleep and generally includes measurement of EEG activity, electroculographic activity and electromyographic activity, as well as other measurements. These results, along with observations, can measure not only sleep latency (the amount of time required to fall asleep), but also sleep continuity (overall balance of sleep and wakefulness) and sleep consolidation (percent of sleeping time spent in delta-wave or restorative sleep) which may be an indication of the quality of sleep.

There are five distinct sleep stages, which can be measured by polysomnography: rapid eye movement (REM) sleep and four stages of non-rapid eye movement (NREM) sleep (stages 1, 2, 3 and 4). Stage 1 NREM sleep is a transition from wakefulness to sleep and occupies about 5% of time spent asleep in healthy adults. Stage 2 NREM sleep, which is characterized by specific EEG waveforms (sleep spindles and K complexes), occupies about 50% of time spent asleep. Stages 3 and 4 NREM sleep (also known collectively as slow-wave sleep and delta-wave sleep) are the deepest levels of sleep and occupy about 10-20% of sleep time. REM sleep, during which the majority of vivid dreams occur, occupies about 20-25% of total sleep.

These sleep stages have a characteristic temporal organization across the night. NREM stages 3 and 4 tend to occur in the first one-third to one-half of the night and increase in, duration in response to sleep deprivation. REM sleep occurs cyclically through the night. Alternating with NREM sleep about every 80-100 minutes. REM sleep periods increase in duration toward the morning. Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

In addition, the compositions of the invention can be useful for the treatment of the sleep disorders characterized by excessive daytime sleepiness such as narcolepsy. Inverse agonists at the serotonin 5HT_{2A} receptor improve the quality of sleep at nighttime which can decrease excessive daytime sleepiness.

Accordingly, another aspect of the present invention relates to the therapeutic use of compositions of the present invention for the treatment of Sleep Disorders. The compositions of the present invention include a compound that is a potent inverse agonist at the serotonin 5HT_{2A} receptor and therefore can be effective in the treatment of Sleep Disorders by promoting one or more of the following: reducing the sleep onset latency period (measure of sleep induction), reducing the number of nighttime awakenings, and prolonging the amount of time in delta-wave sleep (measure of sleep quality enhancement and sleep consolidation) without effecting REM sleep. In addition, compositions of the present invention can be effective either as a monotherapy or in combination with sleep inducing agents, for example but not limited to, antihistamines.

6. Diabetic-Related Pathologies:

Although hyperglycemia is the major cause for the pathogenesis of diabetic complications such as diabetic peripheral neuropathy (DPN), diabetic nephropathy (DN) and diabetic retinopathy (DR), increased plasma serotonin concentration in diabetic patients has also been implicated to play a role in disease progression (Pietraszek, M. H., et al. Thrombosis Res. 1992, 66(6), 765-74; and Andrzejewska-Buczko J, et al., Klin Oczna. 1996; 98(2), 101-4). Serotonin is believed to play a role in vasospasm and increased platelet aggregability. Improving microvascular blood flow is able to benefit diabetic complications.

A recent study by Cameron and Cotter in Naunyn Schmiedebergs Arch Pharmacol. 2003 June; 367(6):607-14, used a 5-HT_{2A} antagonist experimental drug AT-1015, and other non-specific 5-HT_{2A} antagonists including ritanserin and sarpogrelate. These studies found that all three drugs were able to produce a marked correction (82.6-99.7%) of a 19.8% sciatic motor conduction deficit in diabetic rats. Similarly, 44.7% and 14.9% reductions in sciatic endoneurial blood flow and saphenous sensory conduction velocity were completely reversed.

In a separate patient study, sarogrelate was evaluated for the prevention of the development or progression of diabetic nephropathy (Takahashi, T., et al., Diabetes Res Clin Pract. 2002 November; 58(2): 123-9). In the trial of 24 months of treatment, sarpogrelate significantly reduced urinary albumin excretion level.

7. Glaucoma

Topical ocular administration of 5-HT2 receptor antagonists result in a decrease in intra ocular pressure (IOP) in monkeys (Chang et al., J. Ocul Pharmacol 1:137-147 (1985)) and humans (Mastropasqua et al., Acta Opthalmol Scand Suppl 224:24-25 (1997)) indicating utility for similar compounds such as 5-HT_{2A} inverse agonists in the treatment of ocular hypertensin associated with glaucoma. The 5-HT2 receptor antagonist ketanserin (Mastropasqua supra) and sarpogrelate (Takenaka et al., Investig Opthalmol V is Sci 36:S734 (1995)) have been shown to significantly lower IOP in glaucoma patients.

8. Progressive Miltifocal Leukoencephalopathy

Progressive multifocal leukoencephalopathy (PML) is a lethal demyelinating disease caused by an opportunistic viral infection of oligodendrocytes in immunocompromised patients. The causative agent is JC virus, a ubiquitous papovavirus that infects the majority of the population before adulthood and establishes a latent infection in the kidney. In immunocompromised hosts, the virus can reactivate and productively infect oligodendrocytes. This previously rare condition, until 1984 reported primarily in persons with underlying lymphoproliferative disorders, is now more common because it occurs in 4% of patients with AIDS. Patients usually present with relentlessly progressive focal neurologic defects, such as hemiparesis or visual field deficits, or with alterations in mental status. On brain MRI, one or more white matter lesions are present; they are hyperintense on T2-weighted images and hypointense on T1-weighted images. There is no mass effect, and contrast enhancement is rare. Diagnosis can be confirmed by brain biopsy, with demonstration of virus by in situ hybridization or immunocytochemistry. Polymerase chain reaction amplification of JC virus sequences from the CSF can confirm diagnosis without the need for biopsy [see, e.g., Antinori et al., Neurology (1997) 48:687-694; Berger and Major, Seminars in Neurology (1999) 19:193-200; and Portegies, et al., Eur. J. Neurol. (2004) 11:297-304]. Currently, there is no effective therapy. Survival after diagnosis is about 3 to 5 months in AIDS patients.

JC virus enters cells by receptor-mediated clathrin-dependent endocytosis. Binding of JC virus to human glial cells (e.g., oligodendrocytes) induces an intracellular signal that is critical for entry and infection by a ligand-inducible clathrin-dependent mechanism [Querbes et al., J Virology (2004) 78:250-256]. Recently, 5HT_{2A} was shown to be the receptor on human glial cells mediating infectious entry of JC virus by clathrin-dependent endocytosis [Elphick et al., Science (2004) 306:1380-1383]. 5-HT_{2A} antagonists, including ketanserin and ritanserin, inhibited JC virus infection of human glial cells. Ketanserin and ritanserin have inverse agonist activity at 5-HT_{2A}.

5-HT_{2A} antagonists including inverse agonists have been contemplated to be useful in the treatment of PML [Elphick et al., Science (2004) 306:1380-1383]. Prophylactic treatment of HIV-infected patients with 5HT_{2A} antagonists is envisioned to prevent the spread of JC virus to the central nervous system and the development of PML. Aggressive therapeutic treatment of patients with PML is envisioned to reduce viral spread within the central nervous system and prevent additional episodes of demyelination.

In some embodiments, methods are provided for treating progressive multifocal leukoencephalopathy in a patient in need of such treatment, comprising administering to the patient a composition comprising the 5HT_{2A} inverse agonist disclosed herein.

### Representative Methods of the Invention:

One aspect of the present invention pertains to methods for treating a 5HT_{2A} disorder in an individual comprising administering to the individual in need thereof a therapeutically effective amount of a pharmaceutical composition as described herein.

One aspect of the present invention pertains to methods for treating a sleep disorder in an individual comprising administering to the individual in need thereof a therapeutically effective amount of a pharmaceutical composition as described herein.

In some embodiments, the sleep disorder is a dyssomnia. In some embodiments, the dyssomnia is selected from psychophysiological insomnia, sleep state misperception, idiopathic insomnia, obstructive sleep apnea syndrome, central sleep apnea syndrome, central alveolar hypoventilation syndrome, periodic limb movement disorder, restless leg syndrome, inadequate sleep hygiene, environmental sleep disorder, altitude insomnia, adjustment sleep disorder, insufficient sleep syndrome, limit-setting sleep disorder, sleep-onset association disorder, nocturnal eating or drinking syndrome, hypnotic dependent sleep disorder, stimulant-dependent sleep disorder, alcohol-dependent sleep disorder, toxin-induced sleep disorder, time zone change (jet lag) syndrome, shift work sleep disorder, irregular sleep-wake pattern, delayed sleep phase syndrome, advanced sleep phase syndrome and non-24-hour sleep-wake disorder.

In some embodiments, the sleep disorder is a parasomnia. In some embodiments, the parasomnia is selected from confusional arousals, sleepwalking and sleep terrors, rhythmic movement disorder, sleep starts, sleep talking and nocturnal leg cramps.

In some embodiments, the sleep disorder is associated with a medical or psychiatric disorder. In some embodiments, the medical or psychiatric disorder is selected from psychoses, mood disorders, anxiety disorders, panic disorders, alcoholism, cerebral degenerative disorders, dementia, parkinsonism, fatal familial insomnia, sleep-related epilepsy, electrical status epilepticus of sleep, sleep-related headaches, sleeping sickness, nocturnal cardiac ischemia, chronic obstructive pulmonary disease, sleep-related asthma, sleep-related gastroesophageal reflux, peptic ulcer disease, fibrositis syndrome, osteoarthritis, rheumatoid arthritis, fibromyalgia and post-surgical sleep disorder.

One aspect of the present invention pertains to methods of treating platelet aggregation in an individual comprising administering to the individual in need of such treatment a therapeutically effective amount of a pharmaceutical composition as described herein.

One aspect of the present invention pertains to methods of treating coronary artery disease, myocardial infarction, transient ischemic attack, angina, stroke, and atrial fibrillation in an individual comprising administering to the individual in need of such treatment a therapeutically effective amount of a pharmaceutical composition as described herein.

One aspect of the present invention pertains to methods for reducing the risk of blood clot formation in an angioplasty or coronary bypass surgery individual comprising administering to the individual in need thereof a therapeutically effective amount of a pharmaceutical composition as described herein.

One aspect of the present invention pertains to methods for reducing the risk of blood clot formation in an individual suffering from atrial fibrillation, comprising administering to the individual in need thereof a therapeutically effective amount of a pharmaceutical composition as described herein.

One aspect of the present invention pertains to methods for treating asthma in an individual comprising administering to the individual in need thereof a therapeutically effective amount of a pharmaceutical composition as described herein.

One aspect of the present invention pertains to methods for treating a symptom of asthma in an individual comprising administering to the individual in need thereof a therapeutically effective amount of a pharmaceutical composition as described herein.

One aspect of the present invention pertains to methods for treating agitation or a symptom thereof in an individual comprising administering to the individual in need thereof a therapeutically effective amount of a pharmaceutical composition as described herein. In some embodiments, the individual is a cognitively intact elderly individual.

One aspect of the present invention pertains to methods for treating agitation or a symptom thereof in an individual suffering from dementia comprising administering to the individual in need thereof a therapeutically effective amount of a pharmaceutical composition as described herein. In some embodiments, the dementia is due to a degenerative disease of the nervous system. In some embodiments, the dementia is Alzheimers disease, Lewy Body, Parkinson's disease or Huntington's disease. In some embodiments, the dementia is due to diseases that affect blood vessels. In some embodiments, the dementia is due to stroke or multi-infarct dementia.

One aspect of the present invention pertains to methods for treating an individual suffering from at least one of the indications selected from behavioral disorder, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia comprising administering to the individual in need thereof a therapeutically effective amount of a pharmaceutical composition as described herein and a dopamine D₂ receptor antagonist. In some embodiments, the dopamine D₂ receptor antagonist is haloperidol.

One aspect of the present invention pertains to methods for treating an individual with infantile autism, Huntington's chorea or nausea and vomiting from chemotherapy or chemotherapeutic antibodies comprising administering to the individual in need thereof a therapeutically effective amount of a pharmaceutical composition as described herein and a dopamine D₂ receptor antagonist. In some embodiments, the dopamine D₂ receptor antagonist is haloperidol.

One aspect of the present invention pertains to methods for treating schizophrenia in an individual comprising administering to the individual in need thereof a therapeutically effective amount of a pharmaceutical composition as described herein and a dopamine D₂ receptor antagonist. In some embodiments, the dopamine D₂ receptor antagonist is haloperidol.

One aspect of the present invention pertains to methods for treating negative symptoms of schizophrenia induced by the administration of haloperidol to an individual suffering from the schizophrenia, comprising administering to the individual in need thereof a therapeutically effective amount of a pharmaceutical composition as described herein. In some embodiments, the dopamine D₂ receptor antagonist or haloperidol and the pharmaceutical composition are administered in separate dosage forms.

One aspect of the present invention pertains to methods for treating a diabetic-related disorder in an individual comprising administering to the individual in need thereof a therapeutically effective amount of a pharmaceutical composition as described herein. In some embodiments, the diabetic-related disorder is diabetic peripheral neuropathy. In some embodiments, the diabetic-related disorder is diabetic nephropathy. In some embodiments, the diabetic-related disorder is diabetic retinopathy.

One aspect of the present invention pertains to methods for the treatment of glaucoma or other diseases of the eye with abnormal intraocular pressure.

One aspect of the present invention pertains to methods for the treatment of progressive multifocal leukoencephalopathy in an individual comprising administering to the individual in need thereof a therapeutically effective amount of a pharmaceutical composition as described herein.

In some embodiments, the individual in need thereof has a lymphoproliferative disorder. In some embodiments, the lymphoproliferative disorder is leukemia or lymphoma. In some embodiments, the leukemia or lymphoma is chronic lymphocytic leukemia, Hodgkin's disease, or the like.

In some embodiments, the individual in need thereof has a myeloproliferative disorder.

In some embodiments, the individual in need thereof has carcinomatosis.

In some embodiments, the individual in need thereof has a granulomatous or inflammatory disease. In some embodiments, the granulomatous or inflammatory disease is tuberculosis or sarcoidosis.

In some embodiments, the individual in need thereof is immunocompromised. In some embodiments, the immunocompromised individual has impaired cellular immunity. In some embodiments, the impaired cellular immunity comprises impaired T-cell immunity.

In some embodiments, the individual in need thereof is infected with HIV. In some embodiments, the HIV-infected individual has a CD4+ cell count of ≦200/mm³. In some embodiments, the HIV-infected individual has AIDS. In some embodiments, the HIV-infected individual has AIDS-related complex (ARC). In certain embodiments, ARC is defined as the presence of two successive CD4+ cell counts below 200/mm³ and at least two of the following signs or symptoms: oral hairy leukoplakia, recurrent oral candidiasis, weight loss of at least 2.5 kg or 10% of body weight within last six months, multidermatomal herpes zoster, temperature above 38.5° C. for more than 14 consecutive days or more than 15 days in a 30-day period, or diarrhea with more than three liquid stools per day for at least 30 days [see, e.g., Yamada et al., Clin. Diagn. Virol. (1993) 1:245-256].

In some embodiments, the individual in need thereof is undergoing immunosuppressive therapy. In some embodiments, the immunosuppressive therapy comprises administering an immunosuppressive agent [see, e.g., Mueller, Ann Thorac Surg (2004) 77:354-362; and Krieger and Emre, Pediatr Transplantation (2004) 8:594-599]. In some embodiments, the immunosuppressive therapy comprises administering an immunosuppressive agent selected from the group consisting of: corticosteroids (for example, prednisone and the like), calcineurin inhibitors (for example, cyclosporine, tacrolimus, and the like), antiproliferative agents (for example, azathioprine, mycophenolate mofetil, sirolimus, everolimus, and the like), T-cell depleting agents (for example, OKT® 3 monoclonal antibody (mAb), anti-CD3 immunotoxin FN18-CRM9, Campath-1H (anti-CD52) mAb, anti-CD4 mAb, anti-T cell receptor mAb, and the like), anti-IL-2 receptor (CD25) mAb (for example, basiliximab, daclizumab, and the like), inhibitors of co-stimulation (for example, CTLA4-Ig, anti-CD154 (CD40 ligand) mAb, and the like), deoxyspergualin and analogs thereof (for example, 15-DSG, LF-08-0299, LF14-0195, and the like), leflunomide and analogs thereof (for example, leflunomide, FK778, FK779, and the like), FTY720, anti-alpha-4-integrin monoclonal antibody, and anti-CD45 RB monoclonal antibody. In some embodiments, the immunosuppressive agent and the compound or pharmaceutical composition are administered in separate dosage forms. In some embodiments, the immunosuppressive agent and the compound or pharmaceutical composition are administered in a single dosage form.

In some embodiments, the individual in need thereof is undergoing immunosuppressive therapy after organ transplantation. In some embodiments, the organ is liver, kidney, lung, heart, or the like [see, e.g., Singh et al., Transplantation (2000) 69:467-472].

In some embodiments, the individual in need thereof is undergoing treatment for a rheumatic disease. In some embodiments, the rheumatic disease is systemic lupus erythematosus or the like.

In some embodiments, the pharmaceutical composition inhibits JC virus infection of human glial cells.

If desired, the compositions of the present invention may further comprise conventional pharmaceutical additives such as co-surfactants (for example, sodium lauryl sulfate), coloring agents, flavoring agents, fragrances, preserving agents, stabilizers, anti-oxidant and/or thickening agents.

It is noted that when the pharmaceutical compositions described herein are not only intended for use in humans but also in other non-human mammals as well. Indeed, recent advances in the area of animal health-care suggests that consideration be given for the use of active agents, such as 5-HT_{2A} receptor modulators, for the treatment of a 5HT_{2A} mediated disease or disorder in domestic animals (e.g., cats and dogs) and in other domestic animals (e.g., such as cows, chickens, fish, etc.). Those of ordinary skill in the art are readily credited with understanding the utility of such compounds in such settings.

The pharmaceutical compositions of the present invention will be better understood in connection with the following examples, which are intended as an illustration of and not a limitation upon the scope of the invention. Without further elaboration, it is believed that one skilled in the art can, using the preceding description and the information provided in the examples below, practice the present invention to its fullest extent.

### EXAMPLES

### Example 1

### Solubility determination for 1-[3-(4-bromo-2-methyl-2H-pyrazol-3-yl)-4-methoxy-phenyl]-3-(2,4-difluorophenyl)-urea in selected excipients

To 1 mL glass vials was added an excess amount of 1-[3-(4-bromo-2-methyl-2H-pyrazol-3-yl)-4-methoxy-phenyl]-3-(2,4-difluoro-phenyl)-urea and the excipient that resulted in a suspension. For some excipients, a suspension was not observed and therefore the solubility was assumed to be greater than the added amount of the specific volume, for example, Transcutol™ P, PEG 300, PEG 600, Tween™ 20, and Softigen 767 (See Table below). The vial contents were mixed for 30 seconds using a VWR mini vortexter followed by sonication (Branson 1510) for 1 minutes. Vials were placed into a constant temperature bath (i.e., about 25° C.) and allowed to equilibrate for no less than 12 hours. The resulting suspensions were transferred to eppendorf tubes each equipped with a 0.2 μm nylon filter (Costar 8168) and were centrifuged for 10 minutes at 14,000 rpms. The supernatant from each eppendorf tube was collected and diluted with HPLC grade acetonitrile or methonal with appropriate dilution factor. Each solution was analyzed by an HPLC method.

As an example, the following HPLC assay was used to determine the aqueous solubility of 1-[3-(4-bromo-2-methyl-2H-pyrazol-3-yl)-4-methoxy-phenyl]-3-(2,4-difluoro-phenyl)-urea:

HPLC System: Waters 2795; Stationary phase: Xterra® column, MS C18, 3.5 μm, 4.6×50 mm; Mobile phase: Line A: 100% De-ionized-Millipore water; Line B: 1.0% NH₄OH; Line C: 100% HPLC grade Acetonitrile; Gradient: A: 80% to 0% in 8 minute; B: 10% constant in 8 minute; C: 10% to 90% in 8 minute; Flow rate: 1.50 mL/min; Column temperature: 40° C.±5° C.; Sample temperature: 25° C.±5° C.

Photodiode Array Detector: Waters 2996 with UV lamp: 3D data collection; Start wavelength: 210 nm; end wavelength: 320 nm.

Processing wavelength: 220 nm.

Quantification was effected by comparison of the HPLC peak area for each test solution with the peak area taken from a standard plot of concentration versus peak area for standards of known concentration. As is conventional, the standard concentrations for 1-[3-(4-bromo-2-methyl-2H-pyrazol-3-yl)-4-methoxy-phenyl]-3-(2,4-difluoro-phenyl)-urea were selected to fall within a linear range of concentration versus absorbance for the UV detector employed. The standard concentration for 1-[3-(4-bromo-2-methyl-2H-pyrazol-3-yl)-4-methoxy-phenyl]-3-(2,4-difluoro-phenyl)-urea was diluted in serial fashion to obtain a calibration curve. Dilution was effected by adding the acetonitrile from mobile phase. Each saturated equilibrium solution for 1-[3-(4-bromo-2-methyl-2H-pyrazol-3-yl)-4-methoxy-phenyl]-3-(2,4-difluoro-phenyl)-urea obtained after filtering the test vial solutions was diluted with acetonitrile or methonal with appropriate dilution factor to reach the linear range of the standard plot.

The observed solubilities for a variety of excipients are shown in the following table.

### Solubility of 1-[3-(4-bromo-2-methyl-2H-pyrazol-3-yl)-4-methoxy-phenyl]-3-(2,4-difluoro-phenyl)-urea at 25° C. in a Variety of Excipients

| | Solubility |
|---|---|
| Solvent(s) | (mg/mL) |
| Deionized water | <0.01 |
| 0.01 N HCl (pH 2) | <0.01 |
| 25 mM Phosphate buffer (pH 7) | <0.01 |
| Saline (0.9% NaCl solution) | <0.01 |
| 50% Captisol ™ | 0.4 |
| 5% Gum Arabic | <0.01 |
| 1% Tween ™ 80 | 0.4 |
| 0.05% Tween ™ 80 & 0.5% Methocel ™ | <0.01 |
| Transcutol ™ P | >200 |
| PEG 300 | >150 |
| PEG 400 | 376.6 |
| PEG 600 | >325 |
| Labrasol ™ | 348.0 |
| Cremophor ™ RH 40 | 195.7 |
| Tween ™ 80 | 320.2 |
| Tween ™ 20 | >160 |
| Labrafil ™ M 1944 CS | 29.92 |
| Labrafil ™ M 2125 CS | 32.7 |
| Softigen 767 | >200 |

### Example 2

### Pharmacokinetics of 1-[3-(4-bromo-2-methyl-2H-pyrazol-3-yl)-4-methoxy-phenyl]-3-(2,4-difluoro-phenyl)-urea in Healthy Male and Female Volunteers Following Oral Administration

Plasma pharmacokinetics for 1-[3-(4-bromo-2-methyl-2H-pyrazol-3-yl)-4-methoxy-phenyl]-3-(2,4-difluoro-phenyl)-urea were determined in healthy male and female volunteers following oral administration of a capsule formulated dose of 1-[3-(4-bromo-2-methyl-2H-pyrazol-3-yl)-4-methoxy-phenyl]-3-(2,4-difluoro-phenyl)-urea and Cremephor RH40.

### Single oral dose of 1-[3-(4-bromo-2-methyl-2H-pyrazol-3-yl)-4-methoxy-phenyl]-3-(2,4-difluoro-phenyl)-urea in Healthy Male and Female Volunteers

| | | |
|---|---|---|
| AUC (INF) | Cmax | |
| (hr * μg/mL) | (μg/mL) | Tmax (hr) |

| Dose (mg) | Mean | SD | Mean | SD | Mean | SD |
|---|---|---|---|---|---|---|
| 10 | 0.051 | 0.022 | 0.018 | 0.011 | 1.2 | 0.4 |
| 20 | 0.181 | 0.089 | 0.058 | 0.034 | 1.2 | 0.3 |
| 40 | 0.395 | 0.268 | 0.113 | 0.060 | 1.5 | 0.6 |
| 80 | 0.612 | 0.399 | 0.118 | 0.127 | 1.2 | 0.7 |
| 160 | 0.739 | 0.330 | 0.125 | 0.059 | 1.5 | 1.1 |

### Example 3

### Pharmacokinetics of 1-[3-(4-bromo-2-methyl-2H-pyrazol-3-yl)-4-methoxy-phenyl]-3-(2,4-difluoro-phenyl)-urea in male Sprague-Dawley Rats After Oral Administration in Various Excipients

Plasma pharmacokinetics for 1-[3-(4-bromo-2-methyl-2H-pyrazol-3-yl)-4-methoxy-phenyl]-3-(2,4-difluoro-phenyl)-urea were determined in male Sprague-Dawley rats following a 10 mg/kg oral administration of 1-[3-(4-bromo-2-methyl-2H-pyrazol-3-yl)-4-methoxy-phenyl]-3-(2,4-difluoro-phenyl)-urea. 1-[3-(4-Bromo-2-methyl-2H-pyrazol-3-yl)-4-methoxy-phenyl]-3-(2,4-difluoro-phenyl)-urea was formulated as an aqueous suspension, in polyethylene glycol 400 (PEG400), Labrasol, Cremaphor RH40, 80% Tween80 and 20% water (Tween), Cremaphor RH40:Labrasol (1:1, v/v), 40% hydroxypropyl-β-cyclodextrin (HPCD), and dimethylacetamide (DMAC). Dose formulations were administered via oral gavage tube.

### Average Pharmacokinetic Parameters for Male Sprague-Dawley Rats Administered a 10 mg/kg Oral Dose of 1-[3-(4-bromo-2-methyl-2H-pyrazol-3-yl)-4-methoxy-phenyl]-3-(2,4-difluoro-phenyl)-urea

| | AUC (inf) | | Cmax | | | |
|---|---|---|---|---|---|---|
| | (hr * ug/mL) | | (ug/mL) | | Tmax (hr) | |
| Formulation | Mean | Std | Mean | Std | Mean | Std |
| Aqueous | 0.384 | 0.165 | 0.021 | 0.011 | 1.5 | 0.1 |
| Suspension | | | | | | |
| PEG400 | 2.509 | 0.448 | 0.490 | 0.089 | 3.0 | 1.7 |
| Labrasol | 2.208 | 0.271 | 0.297 | 0.048 | 1.7 | 0.6 |
| Tween | 3.180 | 0.714 | 0.904 | 0.194 | 1.3 | 0.6 |
| Cremaphor | 3.784 | 0.433 | 0.768 | 0.156 | 2.3 | 1.5 |
| Cremaphor:Labrasol | 3.015 | 0.355 | 0.302 | 0.052 | 8.0 | 0.0 |
| (1:1, v/v) | | | | | | |
| HPCD¹ | 3.418 | 1.369 | 0.561 | 0.409 | 1.8 | 1.9 |
| DMAC | 3.336 | 0.258 | 0.519 | 0.133 | 2.7 | 1.2 |

¹hydroxypropyl-β-cyclodextrin
²dimethylacetamide

### Example 4

### Pharmacokinetic comparison of 1-[3-(4-bromo-2-methyl-2H-pyrazol-3-yl)-4-methoxy-phenyl]-3-(2,4-difluoro-phenyl)-urea Capsule Dosage Forms in Cynomolgus Monkeys

Plasma pharmacokinetics for 1-[3-(4-bromo-2-methyl-2H-pyrazol-3-yl)-4-methoxy-phenyl]-3-(2,4-difluoro-phenyl)-urea were determined in male cynomolgus monkeys following 30 mg (1-[3-(4-bromo-2-methyl-2H-pyrazol-3-yl)-4-methoxy-phenyl]-3-(2,4-difluoro-phenyl)-urea) oral solution dose (PEG400) and oral administration of 10, 30, and 80 mg (1-[3-(4-bromo-2-methyl-2H-pyrazol-3-yl)-4-methoxy-phenyl]-3-(2,4-difluoro-phenyl)-urea) in two capsule dosage forms. Monkeys were separated into 7 groupings with 3 animals per group. The following dosage forms were compared: capsule dose of 1-[3-(4-bromo-2-methyl-2H-pyrazol-3-yl)-4-methoxy-phenyl]-3-(2,4-difluoro-phenyl)-urea formulated in Cremephor RH 40, capsule dose of 1-[3-(4-bromo-2-methyl-2H-pyrazol-3-yl)-4-methoxy-phenyl]-3-(2,4-difluoro-phenyl)-urea formulated in polyethylene glycol 4000, and a solution dose of 1-[3-(4-bromo-2-methyl-2H-pyrazol-3-yl)-4-methoxy-phenyl]-3-(2,4-difluoro-phenyl)-urea in PEG 400.

### Average Pharmacokinetic Parameters for 1-[3-(4 bromo-2-methyl-2H-pyrazol-3-yl)-4-methoxy-phenyl]-3-(2,4-difluoro-phenyl)-urea After Oral Administration to Male Cynomolgus Monkeys

| | | AUC (inf) | | Cmax | | | |
|---|---|---|---|---|---|---|---|
| Dose | | (hr * ug/mL) | | (ug/mL) | | Tmax (hr) | |
| (mg) | Formulation | Mean | Std | Mean | Std | Mean | Std |
| 10 | Cremephor¹ | 0.291 | 0.05 | 0.087 | 0.039 | 2.3 | 0.6 |
| 30 | Cremephor¹ | 1.525 | 0.602 | 0.451 | 0.226 | 1.7 | 0.6 |
| 80 | Cremephor¹ | 2.968 | 2.107 | 0.502 | 0.334 | 2.3 | 0.3 |
| 10 | PEG4000¹ | 0.166 | 0.092 | 0.009 | 0.005 | 4.0 | 3.5 |
| 30 | PEG4000¹ | 0.334 | 0.224 | 0.026 | 0.021 | 3.8 | 5.3 |
| 80 | PEG4000¹ | 1.188 | 0.446 | 0.089 | 0.033 | 6.0 | 3.5 |
| 30 | PEG400² | 1.082 | 0.511 | 0.186 | 0.103 | 2.3 | 1.2 |

¹Formulated capsule dose
²Solution dose via oral gavage

Those skilled in the art will recognize that various modifications, additions, substitutions, and variations to the illustrative examples set forth herein can be made without departing from the spirit of the invention and are, therefore, considered within the scope of the invention. All documents referenced above, including, but are not limited to, printed publications, and provisional and regular patent applications, are incorporated herein by reference in their entirety.

1. A pharmaceutical composition comprising:
1-[3-(4-bromo-2-methyl-2H-pyrazol-3-yl)-4-methoxy-phenyl]-3-(2,4-difluoro-phenyl)-urea; and
at least one pharmaceutical excipient selected from a glycofurol, a poloxamer, a polyethylene glycol, a polyoxyethylene alkyl ether, a polyoxyethylene oil, a polyoxyethylene sorbitan fatty acid ester, an acyl polyoxyethylene, a polyglycolyzed glyceride, and a hydroxyacyl polyoxyethylene.

2. The pharmaceutical composition according to claim 1, comprising:
a first pharmaceutical excipient that is a polyoxyethylene oil; and
a second pharmaceutical excipient selected from diethylene glycol monoethyl ether, PEG 300, PEG 400, PEG 600, PEG 8 caprylic/capric glycerides, polysorbate 80, polysorbate 20, PEG 300 oleic glycerides, PEG 300 linoleic glycerides, and PEG 300 caprylic/capric glycerides.

3. The pharmaceutical composition according to claim 1, wherein said polyoxyethylene oil is a polyoxyethylene hydrogenated castor oil.

4. The pharmaceutical composition according to claim 3, wherein said polyoxyethylene hydrogenated castor oil is polyoxyl 40 hydrogenated castor oil.

5. The pharmaceutical composition according to claim 1, comprising:
a first pharmaceutical excipient that is a polyglycolyzed glyceride; and
a second pharmaceutical excipient selected from diethylene glycol monoethyl ether, PEG 300, PEG 400, PEG 600, polyoxyl 40 hydrogenated castor oil, polysorbate 80, polysorbate 20, PEG 300 oleic glycerides, PEG 300 linoleic glycerides, and PEG 300 caprylic/capric glycerides.

6. The pharmaceutical composition according to claim 1, wherein said polyglycolyzed glyceride is PEG 8 caprylic/capric glycerides.

7. The pharmaceutical composition according to claim 1, comprising a polyoxyethylene oil and a polyglycolyzed glyceride.

8. The pharmaceutical composition according to claim 1, comprising polyoxyl 40 hydrogenated castor oil and PEG 8 caprylic/capric glycerides.

9. The pharmaceutical composition according to claim 1, comprising polyoxyl 40 hydrogenated castor oil and PEG 8 caprylic/capric glycerides together in an amount of at least about 60% by weight of the total composition.

10. The pharmaceutical composition according to claim 1, comprising polyoxyl 40 hydrogenated castor oil and PEG 8 caprylic/capric glycerides together in an amount of at least about 85% by weight of the total composition.

11. The pharmaceutical composition according to claim 1, comprising polyoxyl 40 hydrogenated castor oil and PEG 8 caprylic/capric glycerides in a ratio of from about 1:9 to about 9:1 by weight.

12. The pharmaceutical composition according to claim 1, comprising polyoxyl 40 hydrogenated castor oil and PEG 8 caprylic/capric glycerides in a ratio of about 1:1 by weight.

13. The pharmaceutical composition according to claim 1, wherein said 1-[3-(4-bromo-2-methyl-2H-pyrazol-3-yl)-4-methoxy-phenyl]-3-(2,4-difluoro-phenyl)-urea is in an amount of about 40% to about 0.001% by weight of the total composition.

14. The pharmaceutical composition according to claim 1, wherein said 1-[3-(4-bromo-2-methyl-2H-pyrazol-3-yl)-4-methoxy-phenyl]-3-(2,4-difluoro-phenyl)-urea is in an amount of about 10% to about 0.001% by weight of the total composition.

15. The pharmaceutical composition according to claim 1, comprising:
1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and
a mixture of polyoxyl 40 hydrogenated castor oil and PEG 8 caprylic/capric glycerides in a ratio of about 1:9 to about 9:1 by weight.

16. The pharmaceutical composition according to claim 1, comprising:
1-[3-(4-bromo-2-methyl-2H-pyrazol-3-yl)-4-methoxy-phenyl]-3-(2,4-difluoro-phenyl)-urea in an amount of about 40% to about 0.001% by weight of the total composition; and
a mixture of polyoxyl 40 hydrogenated castor oil and PEG 8 caprylic/capric glycerides in a ratio of about 1:1 by weight.

17. The pharmaceutical composition according to claim 15, wherein:
1-[3-(4-bromo-2-methyl-2H-pyrazol-3-yl)-4-methoxy-phenyl]-3-(2,4-difluoro-phenyl)-urea is in an amount of about 30% to about 0.001% by weight of the total composition; and
said mixture of polyoxyl 40 hydrogenated castor oil and PEG 8 caprylic/capric glycerides is in an amount of at least about 70% by weight of the total composition.

18. The pharmaceutical composition according to claim 1, wherein said pharmaceutical composition is encapsulated in a soft-gelatin capsule.

19. The pharmaceutical composition according to claim 1, wherein said pharmaceutical composition is suitable for oral administration.

20. The pharmaceutical composition according to claim 1, whereby said pharmaceutical composition provides an AUC of about 30 ng·hr/mL to about 1050 ng·hr/mL after oral administration.

21. The pharmaceutical composition according to claim 1, whereby said pharmaceutical composition provides a Cₘₐₓ of about 10 ng/mL to about 170 ng/mL after oral administration.

22. The pharmaceutical composition according to claim 1, whereby said pharmaceutical composition provides a tₘₐₓ of about 20 minutes to about 130 minutes after oral administration.

23. The pharmaceutical composition according to claim 1, wherein said 1-[3-(4-bromo-2-methyl-2H-pyrazol-3-yl)-4-methoxy-phenyl]-3-(2,4-difluoro-phenyl)-urea is in an amount of about 1 mg to about 160 mg.

24. The pharmaceutical composition according to claim 1, wherein said 1-[3-(4-bromo-2-methyl-2H-pyrazol-3-yl)-4-methoxy-phenyl]-3-(2,4-difluoro-phenyl)-urea is in an amount of about 5 mg, about 10 mg, about 20 mg or about 40 mg.

25. A pharmaceutical composition encapsulated in a soft-gelatin capsule for oral administration consisting essentially of:
1-[3-(4-bromo-2-methyl-2H-pyrazol-3-yl)-4-methoxy-phenyl]-3-(2,4-difluoro-phenyl)-urea in an amount of about 6% to about 0.001% by weight of the total composition; and
a mixture of polyoxyl 40 hydrogenated castor oil and PEG 8 caprylic/capric glycerides in a ratio of about 1:1 by weight, wherein said mixture is in an amount of at least about 94% by weight of the total composition;
whereby said composition after oral administration of about 5 mg dose to about 40 mg dose of 1-[3-(4-bromo-2-methyl-2H-pyrazol-3-yl)-4-methoxy-phenyl]-3-(2,4-difluoro-phenyl)-urea provides an AUC of about 30 ng·hr/mL to about 660 ng·hr/mL, a Cₘₐₓ of about 10 ng/mL to about 245 ng/mL, or a tₘₐₓ of about 20 minutes to about 130 minutes.

26. The pharmaceutical composition according to claim 4, wherein said polyoxyl 40 hydrogenated castor oil is Cremophor® RH 40.

27. The pharmaceutical composition according to claim 2, wherein said PEG 8 caprylic/capric glycerides is Labrasol®.

28. A dosage form comprising:
a) about 0.5 mg to about 500 mg of 1-[3-(4-bromo-2-methyl-2H-pyrazol-3-yl)-4-methoxy-phenyl]-3-(2,4-difluoro-phenyl)-urea; and
b) a polyoxyethylene oil, a polyglycolyzed glyceride, or a mixture thereof.

29. The dosage form according to claim 28, wherein said dosage form comprises polyoxyl 40 hydrogenated castor oil and PEG 8 caprylic/capric glycerides

30. The dosage form according to claim 28, wherein said dosage form comprises polyoxyl 40 hydrogenated castor oil and PEG 8 caprylic/capric glycerides in a ratio of about 1:1 by weight.

31. The dosage form according to claim 28, suitable for oral administration.

32. The dosage form according to claim 29, wherein said polyoxyl 40 hydrogenated castor oil is Cremophor® RH 40, and said PEG 8 caprylic/capric glycerides is Labrasol®g.

33. A method for treating a 5HT_{2A} disorder in an individual comprising administering to said individual in need thereof a therapeutically effective amount of a pharmaceutical composition according to claim 1.

34. A method for treating a sleep disorder in an individual comprising administering to said individual in need thereof a therapeutically effective amount of a pharmaceutical composition according to claim 1.

35. A pharmaceutical composition according to claim 1, for use in a method of treatment of the human or animal body by therapy.

36. A pharmaceutical composition according to claim 1, for use in a method of treatment of a 5HT_{2A} related disorder of the human or animal body by therapy.

37. A pharmaceutical composition according to claim 1, for use in a method of treatment of a sleep disorder of the human or animal body by therapy.

38. A method for preparing a pharmaceutical composition comprising:
1-[3-(4-bromo-2-methyl-2H-pyrazol-3-yl)-4-methoxy-phenyl]-3-(2,4-difluoro-phenyl)-urea; and
a polyoxyethylene oil and a polyglycolyzed glyceride, wherein said method comprises:
dissolving 1-[3-(4-bromo-2-methyl-2H-pyrazol-3-yl)-4-methoxy-phenyl]-3-(2,4-difluoro-phenyl)-urea into said polyoxyethylene oil or said polyglycolyzed glyceride, or a mixture thereof.

39. The method for preparing a pharmaceutical composition according to claim 38, wherein said dissolving is in said polyoxyethylene oil and said polyglycolyzed glyceride in a ratio of about 1:1 by weight.

40. The method for preparing a pharmaceutical composition according to claim 38, wherein said polyoxyethylene oil is polyoxyl 40 hydrogenated castor oil and said polyglycolyzed glyceride is PEG 8 caprylic/capric glycerides.

41. The method for preparing a pharmaceutical composition according to claim 38, wherein said dissolving of 1-[3-(4-bromo-2-methyl-2H-pyrazol-3-yl)-4-methoxy-phenyl]-3-(2,4-difluoro-phenyl)-urea is at a temperature in the range of about 25° C. to about 80° C.

42. The method for preparing a pharmaceutical composition according to claim 38, further comprising the step of filling said pharmaceutical composition in a soft gelatin capsule.

43. The method for preparing a pharmaceutical composition according to claim 40, wherein said polyoxyl 40 hydrogenated castor oil is Cremophor® RH 40, and said PEG 8 caprylic/capric glycerides is Labrasol®.
